(19) United States
(12) Patent Application Publication
(10) Pub. No.: US 2023/0338531 A1
(43) Pub. Date: Oct. 26, 2023

(54) COMPOSITIONS AND USES OF CD45 TARGETED CHIMERIC ANTIGEN RECEPTOR MODIFIED T CELLS

(71) Applicant: City of Hope, Duarte, CA (US)

(72) Inventors: Zhiqiang Wang, Duarte, CA (US); Christine E. Brown, Duarte, CA (US); Stephen J. Forman, Duarte, CA (US)

(21) Appl. No.: 18/021,519

(22) PCT Filed: Aug. 20, 2021
(86) PCT No.: PCT/US2021/046980

§ 371 Date: Feb. 15, 2023

Related U.S. Application Data
(60) Provisional application No. 63/068,289, filed on Aug. 20, 2020.

(51) Int. Cl. A61K 39/00; C07K 14/725; C07K 16/28; C07K 14/73; C07K 14/705; C07K 14/715; A61P 35/00; C12N 15/11; C12N 9/22; C12N 15/90

(52) U.S. Cl. CPC A61K 39/4631; C07K 14/7051; C07K 16/289; C07K 14/70514; C07K 14/70517; C07K 14/70521; C07K 14/7056; C07K 14/7151; C07K 14/70503; A61K 39/4611; A61K 39/4613; A61K 39/4633; A61K 39/4632; A61P 35/00; C12N 15/11; C12N 9/22; C12N 15/907; C07K 2317/622; C07K 2319/03; C07K 2319/02; C12N 2310/20; C12N 2800/80

(57) ABSTRACT

Immune cells, including T cells, expressing a chimeric antigen receptor targeted to CD45 are described. In some cases, the immune cells lack a functional CD45 gene. In some cases, the immune cells also include a modification (a suicide sequence) that allows the cells to be killed in vivo. The immune cells are useful for treating a variety of cancers.

### CLAIM OF PRIORITY

This application claims the benefit of U.S. Provisional Application Ser. No. 63/068,289, filed on Aug. 20, 2020. The entire contents of the foregoing are incorporated herein by reference.

### TECHNICAL FIELD

This disclosure concerns CD45-specific chimeric antigen receptor (CAR)-engineered T cells and NK cells, methods of formulating, and methods of use.

### BACKGROUND

Hematopoietic Cell Transplantation (HCT) is a curative therapeutic option to treat a variety of acquired and inherited malignant and nonmalignant disorders, including hematopoietic malignancies (e.g. Leukemia, lymphoma, and myeloma), genetic and acquired hematopoietic disorders (sickle cell anemia, aplastic anemia and severe combined immunodeficiency) [Stephen J. Forman, R.S.N., Joseph H. Antin, Frederick R. Appelbaum, Thomas' Hematopoietic Cell Transplantation: Stem Cell Transplantation, I, Fifth Edition. 2015; Chabannon, C., et al., Hematopoietic stem cell transplantation in its 60s: A platform for cellular therapies. Sci Transl Med, 2018. 10 (436)]. The preparative regimen is a key element in HCT procedure, which was typically classified into myeloablative, reduced intensity conditioning (RIC) and non-myeloablative regimens. The purpose of preparative regimen is to ablate or reduce recipient bone marrow cells including myeloid cells and hematopoietic stem cells (HSCs) to ensure the engraftment success. Most preparative regimens consist of total body irradiation (TBI), chemotherapy agents or combination of them [Stephen J. Forman, R.S.N., Joseph H. Antin, Frederick R. Appelbaum, Thomas' Hematopoietic Cell Transplantation: Stem Cell Transplantation, I, Fifth Edition. 2015; Gyurkocza, B. and B. M. Sandmaier, Conditioning regimens for hematopoietic cell transplantation: one size does not fit all. Blood, 2014. 124(3):344-53]. These regimens all have side effects in addition to myelotoxicity, which may lead to severe and even life-threatening complications. Novel regimens such as radiolabeled, drug conjugated or cold monoclonal antibodies targeting CD33, CD133, c-kit and CD45 were explored to reduce the toxicity and achieved promising results [Appelbaum, F. R., et al., The use of radiolabeled anti-CD33 antibody to augment marrow irradiation prior to marrow transplantation for acute myelogenous leukemia. Transplantation, 1992. 54(5):829-33; Matthews, D. C., et al., Phase I study of (131)I-anti-CD45 antibody plus cyclophosphamide and total body irradiation for advanced acute leukemia and myelodysplastic syndrome. Blood, 1999. 94(4):1237-47; Pagel, J. M., et al., 131I-anti-CD45 antibody plus busulfan and cyclophosphamide before allogeneic hematopoietic cell transplantation for treatment of acute myeloid leukemia in first remission. Blood, 2006. 107(5):2184-91; Green, D. J., et al., Pretargeting CD45 enhances the selective delivery of radiation to hematolymphoid tissues in nonhuman primates. Blood, 2009. 114(6):1226-35; Mawad, R., et al., Radiolabeled anti-CD45 antibody with reduced-intensity conditioning and allogeneic transplantation for younger patients with advanced acute myeloid leukemia or myelodysplastic syndrome. Biol Blood Marrow Transplant, 2014. 20(9):1363-8]. However, concerns regarding stromal cell ablation by high dose of irradiation or toxin in bone marrow and the sophisticated requirements of radioisotope reagent administration limited some of the applications. Recently, Chimeric Antigen Receptor (CAR) T cells targeting c-Kit were explored on animal model to be used as HCT conditioning method and demonstrated promising BM ablation effects [Chabannon, C., et al., Hematopoietic stem cell transplantation in its 60s: A platform for cellular therapies. Sci Transl Med, 2018. 10(436)]. However, c-Kit is widely expressed on several key organs in healthy human body such as lung, brain and skin [Lammie, A., et al., Expression of c-kit and kit ligand proteins in normal human tissues. J Histochem Cytochem, 1994. 42(11):1417-25; Miettinen, M. and J. Lasota, KIT (CD117): a review on expression in normal and neoplastic tissues, and mutations and their clinicopathologic correlation. Appl Immunohistochem Mol Morphol, 2005. 13(3):205-20.] and on-target off-site toxicity would be a concern by CAR T targeting.

CD45 is a protein tyrosine phosphatase encoded by PTPRC gene (Human CD45: GenBank ID 5788). CD45 is exclusively expressed on majority of hematopoietic lineage cells including T cells, B cells, myeloid cells, and HSC with exception of erythrocytes and platelets [Rheinlander, A., B. Schraven, and U. Bommhardt, CD45 in human physiology and clinical medicine. Immunol Lett, 2018. 196:22-32; Bhatia, M., et al., Purification of primitive human hematopoietic cells capable of repopulating immune-deficient mice. Proc Natl Acad Sci U S A, 1997. 94(10):5320-5]. CD45 is also widely expressed on different hematopoietic malignant cells as well as cancer stem cells in diseases such as AML, CML, ALL, MM, etc. [Bonnet, D. and J. E. Dick, Human acute myeloid leukemia is organized as a hierarchy that originates from a primitive hematopoietic cell. Nat Med, 1997. 3(7):730-7; Dick, J. E., Stem cell concepts renew cancer research. Blood, 2008. 112(13):4793-807], which make CAR T cells targeting CD45 a potential therapy against these hematopoietic malignancies. Since CD45 is also expressed on T cells, fratricide effect of CD45CAR T cells is expected and presents a significant hurdle.

### SUMMARY

Described herein are methods for making and using CD45KO CD45 targeted CAR T cells (also herein called CD45 CAR T cells) and CD45KO CD45 targeted CAR natural killer (NK) cells (also herein called CD45 CAR NK cells) to treat a variety of acquired and inherited malignant and nonmalignant disorders, for example, hematopoietic malignancies (e.g. Leukemia, lymphoma, and myeloma), genetic and acquired hematopoietic disorders (sickle cell anemia, aplastic anemia and severe combined immunodeficiency). In some embodiments, CD45 (encoded by the gene PTPRC) is knocked out, knocked down, or mutated (e.g., by gene editing technologies such as CRISPR-Cas9 or TALEN system). In some embodiments, CD45KO CD45 CAR T cells were generated by sequential gene editing followed by CAR transduction. The CD45KO CD45 CAR T cells possess potent antigen-specific anti-tumor efficacy in vitro and in vivo, as well as myeloid & lymphoid depletion capability in vitro.

In some embodiments, described herein is a method of treating a hematopoietic malignancy or hematopoietic disorder. In some embodiments, the hematopoietic malignancy or hematopoietic disorder is any one or more of a leukemia, a lymphoma, a myeloma, a myeloid leukemia, a T cell leukemia, a T cell lymphoma, a B cell leukemia, a B cell lymphoma, AML, CML, ALL, multiple myeloma, sickle cell anemia, aplastic anemia, and severe combined immunodeficiency. Also described herein is a method of treating CD45-positive cancers (including, e.g., peripheral T cell lymphoma, adult T cell lymphoma, anaplastic large cell lymphoma, primary cutaneous T cell lymphoma, renal cell carcinoma, lung cancer, hepatocellular carcinoma, and diffuse large B-cell lymphoma) in a patient comprising administering a population of autologous or allogeneic human T cells transduced by a vector comprising a nucleic acid molecule described herein, wherein the T cell leukemia, the T cell lymphoma, the B cell leukemia, and or the B cell lymphoma comprises cells expressing CD45. In various embodiments: the chimeric antigen receptor or polypeptide is administered locally or systemically; the CD45-expressing cells are cancerous T cells; and the chimeric antigen receptor or polypeptide is administered by single or repeat dosing.

Also described herein are methods for using CD45 CAR T cells or CD45 CAR NK cells as anti-cancer agents selective against CD45-positive cells, also described herein are methods of decreasing the population of non-cancerous CD45-positive cells. In some embodiments, described herein is a method of reducing or eliminating CD45-positive cells in a subject comprising administering a population of autologous or allogeneic human T or NK cells transduced by a vector comprising the nucleic acid molecule encoding a CD45 CAR or a CD45 polypeptide, wherein CD45 (PTPRC) is knocked out, knocked down, or mutated in the human T or NK cells.

Also described herein is a method of hematological cell transplantation conditioning. In some embodiments, hematological cell transplantation conditioning in a patient comprises administering a population of autologous or allogeneic human T or NK cells transduced by a vector comprising the nucleic acid molecule encoding a CD45 CAR or a CD45 polypeptide, wherein the PTPRC is knocked out, knocked down, or mutated in the T or NK cells.

Described herein is a nucleic acid molecule comprising a nucleotide sequence encoding a chimeric antigen receptor (CAR) or polypeptide, wherein the chimeric antigen receptor or polypeptide comprises: an scFv targeting CD45, a spacer, a transmembrane domain, a co-stimulatory domain, and a CD3 ζ signaling domain.

In various embodiments: the transmembrane domain is selected from: a CD4 transmembrane domain or variant thereof having 1-5 amino acid modifications, a CD8 transmembrane domain or variant thereof having 1-5 amino acid modifications, a CD28 transmembrane domain or a variant thereof having 1-5 amino acid modifications; the spacer comprises 20-150 amino acids and is located between the scFv and the transmembrane domain; the transmembrane domain is a CD4 transmembrane domain or variant thereof having 1-5 amino acid modifications; the transmembrane domain is a CD4 transmembrane domain; the chimeric antigen receptor comprises a transmembrane domain selected from: a CD4 transmembrane domain or variant thereof having 1-2 amino acid modifications, a CD8 transmembrane domain or variant thereof having 1-2 amino acid modifications, a CD28 transmembrane domain or a variant thereof having 1-2 amino acid modifications; the spacer region comprises an amino acid sequence selected from the group consisting of SEQ ID NOs: 2-12 or a variant thereof having 1-5 amino acid modifications; the spacer comprises an IgG hinge region; the spacer comprises 10-50 amino acids; the costimulatory domain comprises the amino acid sequence of SEQ ID NO: 22, 23, or 24 or a variant thereof having 1-5 amino acid modifications; the CD3ζ signaling domain comprises the amino acid sequence of SEQ ID NO:21; a linker of 3 to 15 amino acids is located between the costimulatory domain and the CD3 ζ signaling domain or variant thereof, the CAR or polypeptide comprises the amino acid sequence of SEQ ID NO: 29 or a variant thereof having 1-5 amino acid modifications; the scFv comprises the amino acid sequence of SEQ ID NO:1; the nucleic acid molecule of claim 1.

Also disclosed herein is: a viral vector comprising a nucleic acid molecule described herein; a population of human T cells (e.g., a population comprising central memory T cells) or of human NK cells transduced by a vector comprising a nucleic acid molecule described herein.

In some embodiments, the T cells comprise PBMC, dPBMC (PBMC with depletion of CD14+ and CD25+ cells), Tn/mem (naïve and memory T cells, CD62L+ enriched from dPBMC), or Tcm (central memory T cells).

In various embodiments: the chimeric antigen receptor or polypeptide comprises: a CD45 scFv, e.g., an scFv comprising the amino acid sequence QVQLVESGGGLVQPGGSLKLSCAASGFDFSRYWMSWVRQAPGKGLEWIGEINPTSS TINFTPSLKDKVFISRDNAKNTLYLQMSKVRSEDTALYYCARGNYYRYGDAMDYW GQGTSVTVSKISGGGGSGGGGSGGGGSGGGGSGGGGSSDIVLTQSPASLAVSLGQRA TISCRASKSVSTSGYSYLHWYQQKPGQPPKLLIYLASNLESGVPARFSGSGSGTDFTL NIHIPVEEEDAATYYCQHSRELPFTFGSGTKLEIK (SEQ ID NO:1) with up to 5 or up to 10 single amino acid substitutions).

In certain embodiments, the CD45 scFv comprises a heavy chain variable region that is at least 95% identical to or includes up to 5 single amino acid substitutions compared to:

(SEQ ID NO: 32)
QVQLVESGGGLVQPGGSLKLSCAASGFDFSRYWMSWVRQAPGKGLEWIGE
INPTSSTINFTPSLKDKVFISRDNAKNTLYLQMSKVRSEDTALYYCARGN
YYRYGDAMDYWGQGTSVTVSKIS
or
(SEQ ID NO: 46)
QVQLVESGGGLVQPGGSLKLSCAASGFDFSRYWMSWVRQAPGKGLEWIGE
INPTSSTINFTPSLKDKVFISRDNAKNTLYLQMSKVRSEDTALYYCARGN
YYRYGDAMDYWGQGTSVTVSK.

In certain embodiments, the CD45 scFv comprises a heavy chain variable region that comprises a CDR1 comprising: RYWMS (SEQ ID NO: 47), a CDR2 comprising EINPTSSTINFTPSLKD (SEQ ID NO: 48); and a CDR3 comprising GNYYRYGDAMDY (SEQ ID NO: 49). In some embodiments, the CD45 scFv comprises a heavy chain variable region that is at least 95% identical to or includes up to 5 single amino acid substitutions compared to: QVQLVESGGGLVQPGGSLKLSCAASGFDFSRYWMSWVRQAPGKGLEWIGEINPTSS TINFTPSLKDKVFISRDNAKNTLYLQMSKVRSEDTALYYCARGNYYRYGDAMDYW GQGTSVTVSKIS (SEQ ID NO: 32) or QVQLVESGGGLVQPGGSLKLSCAASGFDFSRYWMSWVRQAPGKGLEWIGEINPTSS TINFTPSLKDKVFISRDNAKNTLYLQMSKVRSEDTALYYCARGNYYRYGDAMDYW GQGTSVTVSK (SEQ ID NO: 46) and comprises a CDR1 comprising: RYWMS (SEQ ID NO: 47), a CDR2 comprising EINPTSSTINFTPSLKD (SEQ ID NO: 48); and a CDR3 comprising GNYYRYGDAMDY (SEQ ID NO: 49).

In certain embodiments, the CD45 scFv comprises a light chain variable region that is at least 95% identical to or includes up to 5 single amino acid substitutions compared to:

(SEQ ID NO: 33)
DIVLTQSPASLAVSLGQRATISCRASKSVSTSGYSYLHWYQQKPGQPPKL
LIYLASNLESGVPARFSGSGSGTDFTLNIHPVEEEDAATYYCQHSRELPF
TFGSGTKLEIK.

In certain embodiments, the CD45 scFv comprises a light chain variable region that comprises a CDR1 comprising: RASKSVSTSGYSYLH (SEQ ID NO: 50), a CDR2 comprising LASNLES (SEQ ID NO: 51); and a CDR3 comprising QHSRELPFTFGSGT (SEQ ID NO: 52). In certain embodiments, the CD45 scFv comprises a light chain variable region that is at least 95% identical to or includes up to 5 single amino acid substitutions compared to:

(SEQ ID NO: 33)
DIVLTQSPASLAVSLGQRATISCRASKSVSTSGYSYLHWYQQKPGQPPKL
LIYLASNLESGVPARFSGSGSGTDFTLNIHPVEEEDAATYYCQHSRELPF
TFGSGTKLEIK
and comprises a CDR1 comprising:
(SEQ ID NO: 50)
RASKSVSTSGYSYLH,
a CDR2 comprising
(SEQ ID NO: 51)
LASNLES;
and a CDR3 comprising
(SEQ ID NO: 52)
QHSRELPFTFGSGT.

Also described are T cells or NK cells harboring a vector expressing the CAR or polypeptide. In various embodiments: at least 20%, 30%, or 40% of the transduced human T cells are central memory T cells; at least 30% of the transduced human T cells are CD4+ and CD62L+ or CD8+ and CD62L+; the population of human T cells are autologous to the patient; and the population of human T cells are allogenic to the patient.

Also described herein is a method of preparing CD45 CAR T cells comprising: providing a population of autologous or allogeneic human T cells, knock out, knock down, or mutate the PTPRC gene in the T cells, and transducing the T cells by a vector comprising the nucleic acid molecule encoding a CD45 CAR or a CD45 polypeptide, wherein the T cells comprise PBMC, dPBMC (PBMC with depletion of CD14+ and CD25+ cells), Tn/mem (naïve and memory T cells, CD62L+ enriched from dPBMC), or Tcm (central memory T cells).

CD45 Targeted CAR

The CD45 targeted CAR (also called “CD45 CAR”) or CD45 targeted polypeptide (also called “CD45 polypeptide”) described herein include a CD45 targeting scFv. In some embodiments, an scFv comprising the amino acid sequence: QVQLVESGGGLVQPGGSLKLSCAASGFDFSRYWMSWVRQAPGKGLEWIGEINPTSS TINFTPSLKDKVFISRDNAKNTLYLQMSKVRSEDTALYYCARGNYYRYGDAMDYW GQGTSVTVSKISGGGGSGGGGSGGGGSGGGGSGGGGSSDIVLTQSPASLAVSLGQRA TISCRASKSVSTSGYSYLHWYQQKPGQPPKLLIYLASNLESGVPARFSGSGSGTDFTL NIHIPVEEEDAATYYCQHSRELPFTFGSGTKLEIK (SEQ ID NO:1) or comprising the sequence QVQLVESGGGLVQPGGSLKLSCAASGFDFSRYWMSWVRQAPGKGLEWIGEINPTSS TINFTPSLKDKVFISRDNAKNTLYLQMSKVRSEDTALYYCARGNYYRYGDAMDYW GQGTSVTVSKIS (SEQ ID NO:32) and the sequence DIVLTQSPASLAVSLGQRATISCRASKSVSTSGYSYLHWYQQKPGQPPKLLIYLASNL ESGVPARFSGSGSGTDFTLNIIIPVEEEDAATYYCQHSRELPFTFGSGTKLEIK (SEQ ID NO:33) joined by a flexible linker.

In some embodiments, a useful flexible linker is 1, 2, 3, 4, 5, 6, 7, 8, 9, or 10 repeats of the sequence GGGS (SEQ ID NO:34). In some embodiments, a useful flexible linker is 1, 2, 3, 4, 5, 6, 7, 8, 9, or 10 repeats of the sequence GGGGS (SEQ ID NO:35).

A useful CD45 CAR or CD45 polypeptide can consist of or comprises the amino acid sequence of SEQ ID NO:30, 63, 66, 69, 72, 75, 78, or 81 (mature CAR lacking a signal sequence) or the CD45 CAR or CD45 polypeptide can consist of or comprise the amino acid sequence of SEQ ID NO:29, 62, 65, 68, 71, 74, 77, or 80 (immature CAR having a GMCSFRa signal sequence). The CAR or polypeptide can be expressed in a form that includes a signal sequence, e.g., a human GM-CSF receptor alpha signal sequence (MLLLVTSLLLCELPHPAFLLIP; SEQ ID NO:36). The CAR or polypeptide can be expressed with additional sequences that are useful for monitoring expression or inhibiting CAR expression via an inducible suicide switch, for example, a T2A skip sequence and a truncated EGFR or truncated CD19 (can consist of or comprise the amino acid sequence of SEQ ID NO:31, 64, 67, 70, 73, 76, 79, or 82). Thus, the CAR or polypeptide can comprise or consist of the amino acid sequence of SEQ ID Nos: 1, 29, 30, 31, 62, 63, 64, 65, 66, 67, 68, 69, 70, 71, 72, 73, 74, 75, 76, 77, 78, 79, 80, 81, or 82, or can comprise or consist of an amino acid sequence that is at least 95%, 96%, 97%, 98% or 99% identical to SEQ ID Nos: 1, 29, 30, 31, or 62-82. The CAR or polypeptide can comprise or consist of the amino acid sequence of any of SEQ ID Nos 1, 29, 30, 31, or 62-82 with up to 1, 2, 3, 4 or 5 amino acid changes (preferably conservative amino acid changes). The CAR or polypeptide can comprise SEQ ID NO:32 with up to 1, 2, 3, 4 or 5 amino acid changes (preferably conservative amino acid changes) and SEQ ID NO:33 with up to 1, 2, 3, 4 or 5 amino acid changes (preferably conservative amino acid changes) joined by a flexible linker.

In some embodiments, the nucleic acid encoding amino acid sequences SEQ ID NOs:1, 29-33, and 62-82 are codon optimized.

Spacer Region

The CAR or polypeptide described herein can include a spacer located between the CD45 targeting domain (i.e., a CD45 targeted ScFv or variant thereof) and the transmembrane domain. A variety of different spacers can be used. Some of them include at least portion of a human Fc region, for example a hinge portion of a human Fc region or a CH3 domain or variants thereof. Table 1 below provides various spacers that can be used in the CARs described herein.

TABLE 1
Examples of Spacers

| Name | Length | Sequence |
|---|---|---|
| a3 | 3 aa | AAA |
| linker | 10 aa | GGGSSGGGSG (SEQ ID NO: 2) |
| IgG4 hinge (S→P) | 12 aa | ESKYGPPCPPCP (SEQ ID NO: 3) |
| (S228P) | | |
| IgG4 hinge | 12 aa | ESKYGPPCPSCP (SEQ ID NO: 4) |
| IgG4 hinge (S228P) + linker | 22 aa | ESKYGPPCPPCPGGGSSGGGSG (SEQ ID NO: 5) |
| CD28 hinge | 39 aa | IEVMYPPPYLDNEKSNGTIIHVKGKHLCPSPLFPGPSKP (SEQ ID NO: 6) |
| CD8 hinge-48aa | 48 aa | AKPTTTPAPRPPTPAPTIASQPLSLRPEACRPAAGGAVHTRGLDFACD (SEQ |
| | | ID NO: 7) |
| CD8 hinge-42aa | 42aa | PAPRPPTPAPTIASQPLSLRPEACRPAAGGAVHTRGLDFACD (SEQ ID |
| | | NO: 8) |
| IgG4(HL-CH3) | 129 aa | ESKYGPPCPPCPGGGSSGGGSGGQPREPQVYTLPPSQEEMTKNQVSLTCL |
| Also called IgG4(HL-ΔCH2) | | VKGFYPSDIAVEWESNGQPENNYKTTPPVLDSDGSFFLYSRLTVDKSRWQE |
| (includes S228P in hinge) | | GNVFSCSVMHEALHNHYTQKSLSLSLGK (SEQ ID NO: 9) |
| IgG4(L235E, N297Q) | 229 aa | ESKYGPPCPSCPAPEFEGGPSVFLFPPKPKDTLMISRTPEVTCVVVDVSQED |
| | | PEVQFNWYVDGVEVHNAKTKPREEQFQSTYRVVSVLTVLHQDWLNGKEY |
| | | KCKVSNKGLPSSIEKTISKAKGQPREPQVYTLPPSQEEMTKNQVSLTCLVKG |
| | | FYPSDIAVEWESNGQPENNYKTTPPVLDSDGSFFLYSRLTVDKSRWQEGN |
| | | VFSCSVMHEALHNHYTQKSLSLSLGK (SEQ ID NO: 10) |
| IgG4(S228P, L235E, N297Q) | 229 aa | ESKYGPPCPPCPAPEFEGGPSVFLFPPKPKDTLMISRTPEVTCVVVDVSQED |
| | | PEVQFNWYVDGVEVHNAKTKPREEQFQSTYRVVSVLTVLHQDWLNGKEY |
| | | KCKVSNKGLPSSIEKTISKAKGQPREPQVYTLPPSQEEMTKNQVSLTCLVKG |
| | | FYPSDIAVEWESNGQPENNYKTTPPVLDSDGSFFLYSRLTVDKSRWQEGN |
| | | VFSCSVMHEALHNHYTQKSLSLSLGK (SEQ ID NO: 11) |
| IgG4(CH3) | 107 aa | GQPREPQVYTLPPSQEEMTKNQVSLTCLVKGFYPSDIAVEWESNGQPENN |
| Also called IgG4(ΔCH2) | | YKTTPPVLDSDGSFFLYSRLTVDKSRWQEGNVFSCSVMHEALHNHYTQKSL |
| | | SLSLGK (SEQ ID NO: 12) |

Some spacer regions include all or part of an immunoglobulin (e.g., IgG1, IgG2, IgG3, IgG4) hinge region, i.e., the sequence that falls between the CH1 and CH2 domains of an immunoglobulin, e.g., an IgG4 Fc hinge or a CD8 hinge. Some spacer regions include an immunoglobulin CH3 domain (called CH3 or ΔCH2) or both a CH3 domain and a CH2 domain. The immunoglobulin derived sequences can include one or more amino acid modifications, for example, 1, 2, 3, 4 or 5 substitutions, e.g., substitutions that reduce off-target binding.

The hinge/linker region can also comprise an IgG4 hinge region having the sequence ESKYGPPCPSCP (SEQ ID NO:4) or ESKYGPPCPPCP (SEQ ID NO:3). The hinge/linger region can also comprise the sequence ESKYGPPCPPCP (SEQ ID NO:3) followed by the linker sequence GGGSSGGGSG (SEQ ID NO:2) followed by IgG4 CH3 sequence GQPREPQVYTLPPSQEEMTKNQVSLTCLVKGFYPSDIAVEWESNGQPENNYKTTPPV LDSDGSFFLYSRLTVDKSRWQEGNVFSCSVMHEALHNHYTQKSLSLSLGK (SEQ ID NO:12). Thus, the entire linker/spacer region can comprise the sequence: ESKYGPPCPPCPGGGSSGGGSGGQPREPQVYTLPPSQEEMTKNQVSLTCLVKGFYPS DIAVEWESNGQPENNYKTTPPVLDSDGSFFLYSRLTVDKSRWQEGNVFSCSVMHEA LHNHYTQKSLSLSLGK (SEQ ID NO:11). In some cases, the spacer has 1, 2, 3, 4, or 5 single amino acid changes (e.g., conservative changes) compared to SEQ ID NO:11. In some cases, the IgG4 Fc hinge/linker region that is mutated at two positions (L235E; N297Q) in a manner that reduces binding by Fc receptors (FcRs).

Transmembrane Domain

A variety of transmembrane domains can be used in the. Table 2 includes examples of suitable transmembrane domains. Where a spacer region is present, the transmembrane domain (TM) is located carboxy terminal to the spacer region.

TABLE 2
Examples of Transmembrane Domains

| Name | Accession | Length | Sequence |
|---|---|---|---|
| CD3z | J04132.1 | 21aa | LCYLLDGILFIYGVILTALFL (SEQ ID NO: 13) |
| CD28 | NM_006139 | 27aa | FWVLVVVGGVLACYSLLVTVAFIIFWV (SEQ ID NO: 14) |
| CD28(M) | NM_006139 | 28aa | MFWVLVVVGGVLACYSLLVTVAFIIFWV (SEQ ID NO: 15) |
| CD4 | M35160 | 22aa | MALIVLGGVAGLLLFIGLGIFF (SEQ ID NO: 16) |
| CD8tm | NM_001768 | 21aa | IYIWAPLAGTCGVLLLSLVIT (SEQ ID NO: 17) |
| CD8tm2 | NM_001768 | 23aa | IYIWAPLAGTCGVLLLSLVITLY (SEQ ID NO: 18) |
| CD8tm3 | NM_001768 | 24aa | IYIWAPLAGTCGVLLLSLVITLYC (SEQ ID NO: 19) |
| 41BB | NM_001561 | 27aa | IISFFLALTSTALLFLLFF LTLRFSVV (SEQ ID NO: 20) |
| NKG2D | NM_007360 | 21aa | PFFFCCFIAVAMGIRFIIMVT (SEQ ID NO: 53) |

Costimulatory Domain

The costimulatory domain can be any domain that is suitable for use with a CD3ζ signaling domain. In some cases the co-signaling domain is a 4-1BB co-signaling domain that includes a sequence that is at least 90%, at least 95%, at least 98% identical to or identical to: KRGRKKLLYIFKQPFMRPVQTTQEEDGCSCRFPEEEEGGCEL (SEQ ID NO:24). In some cases, the 4-1BB co-signaling domain has 1, 2, 3, 4 of 5 amino acid changes (preferably conservative) compared to SEQ ID NO:24.

The costimulatory domain(s) are located between the transmembrane domain and the CD3ζ signaling domain. Table 3 includes examples of suitable costimulatory domains together with the sequence of the CD3ζ signaling domain.

TABLE 3
CD3ζ Domain and Examples of Costimulatory Domains

| Name | Accession | Length | Sequence |
|---|---|---|---|
| CD37 | J04132.1 | 113 aa | RVKFSRSADAPAYQQGQNQLYNELNLGRREEYDVLDKRRGR |
| | | | DPEMGGKPRRKNPQEGLYNELQKDKMAEAYSEIGMKGERR |
| | | | RGKGHDGLYQGLSTATKDTYDALHMQALPPR (SEQ ID |
| | | | NO: 21) |
| CD28 | NM_006139 | 42aa | RSKRSRLLHSDYMNMTPRRPGPTRKHYQPYAPPRDFAAYRS |
| | | | (SEQ ID NO: 22) |
| CD28gg* | NM_006139 | 42aa | RSKRSRGGHSDYMNMTPRRPGPTRKHYQPYAPPRDFAAYR |
| | | | S (SEQ ID NO: 23) |
| 41BB | NM_001561 | 42 aa | KRGRKKLLYIFKQPFMRPVQTTQEEDGCSCRFPEEEEGGCEL |
| | | | (SEQ ID NO: 24) |
| OX40 | NM_003327 | 42 aa | ALYLLRRDQRLPPDAHKPPGGGSFRTPIQEEQADAHSTLAKI |
| | | | (SEQ ID NO: 25) |
| 2B4 | NM_016382 | 120 aa | WRRKRKEKQSETSPKEFLTIYEDVKDLKTRRNHEQEQTFPGG |
| | | | GSTIYSMIQSQSSAPTSQEPAYTLYSLIQPSRKSGSRKRNHSPS |
| | | | FNSTIYEVIGKSQPKAQNPARLSRKELENFDVYS (SEQ ID |
| | | | NO: 54) |

In various embodiments: the costimulatory domain is selected from the group consisting of: a costimulatory domain depicted in Table 3 or a variant thereof having 1-5 (e.g., 1 or 2) amino acid modifications, a CD28 costimulatory domain or a variant thereof having 1-5 (e.g., 1 or 2) amino acid modifications, a 4-1BB costimulatory domain or a variant thereof having 1-5 (e.g., 1 or 2) amino acid modifications and an OX40 costimulatory domain or a variant thereof having 1-5 (e.g., 1 or 2) amino acid modifications. In certain embodiments, a 4-1BB costimulatory domain or a variant thereof having 1-5 (e.g., 1 or 2) amino acid modifications in present. In some embodiments there are two costimulatory domains, for example a CD28 co-stimulatory domain or a variant thereof having 1-5 (e.g., 1 or 2) amino acid modifications (e.g., substitutions) and a 4-1BB co-stimulatory domain or a variant thereof having 1-5 (e.g., 1 or 2) amino acid modifications (e.g., substitutions). In various embodiments the 1-5 (e.g., 1 or 2) amino acid modification are substitutions. The costimulatory domain is amino terminal to the CD3ζ signaling domain and a short linker consisting of 2-10, e.g., 3 amino acids (e.g., GGG) is can be positioned between the costimulatory domain and the CD3ζ signaling domain.

CD3ζ Signaling Domain

The CD3ζ Signaling domain can be any domain that is suitable for use with a CD3ζ signaling domain. In some cases, the CD3ζ signaling domain includes a sequence that is at least 90%, at least 95%, at least 98% identical to or identical to: RVKFSRSADAPAYQQGQNQLYNELNLGRREEYDVLDKRRGRDPEMGGKPRRKNPQ EGLYNELQKDKMAEAYSEIGMKGERRRGKGHDGLYQGLSTATKDTYDALHMQAL PPR (SEQ ID NO:21). In some cases, the CD3ζ signaling has 1, 2, 3, 4 of 5 amino acid changes (preferably conservative) compared to SEQ ID NO:21.

Truncated EGFR or CD19

The CD3ζ signaling domain can be followed by a ribosomal skip sequence (e.g., LEGGGEGRGSLLTCGDVEENPGPR; SEQ ID NO:27) and a truncated EGFR having a sequence that is at least 90%, at least 95%, at least 98% identical to or identical to: LVTSLLLCELPHPAFLLIPRKVCNGIGIGEFKDSLSINATNIKHFKNCTSISGDLHILPVA FRGDSFTHTPPLDPQELDILKTVKEITGFLLIQAWPENRTDLHAFENLEIIRGRTKQHG QFSLAVVSLNITSLGLRSLKEISDGDVIISGNKNLCYANTINWKKLFGTSGQKTKIISN RGENSCKATGQVCHALCSPEGCWGPEPRDCVSCRNVSRGRECVDKCNLLEGEPREF VENSECIQCHPECLPQAMNITCTGRGPDNCIQCAHYIDGPHCVKTCPAGVMGENNTL VWKYADAGHVCHLCHPNCTYGCTGPGLEGCPTNGPKIPSIATGMVGALLLLLVVAL GIGLFM (SEQ ID NO:28). In some cases, the truncated EGFR has 1, 2, 3, 4 of 5 amino acid changes (preferably conservative) compared to SEQ ID NO:28. Alternatively the CD3ζ signaling domain can be followed by a ribosomal skip sequence (e.g., LEGGGEGRGSLLTCGDVEENPGPR; SEQ ID NO:27) and a truncated CD19R (also called CD19t) having a sequence that is at least 90%, at least 95%, at least 98% identical to or identical to:

(SEQ ID NO: 26)
MPPPRLLFFLLFLTPMEVRPEEPLVVKVEEGDNAVLQCLKGTSDGPTQQ
LTWSRESPLKPFLKLSLGLPGLGIHMRPLAIWLFIFNVSQQMGGFYLCQ
PGPPSEKAWQPGWTVNVEGSGELFRWNVSDLGGLGCGLKNRSSEGPSSP
SGKLMSPKLYVWAKDRPEIWEGEPPCVPPRDSLNQSLSQDLTMAPGSTL
WLSCGVPPDSVSRGPLSWTHVHPKGPKSLLSLELKDDRPARDMWVMETG
LLLPRATAQDAGKYYCHRGNLTMSFHLEITARPVLWHWLLRTGGWKVSA
VTLAYLIFCLCSLVGILHLQRALVLRRKR

An amino acid modification refers to an amino acid substitution, insertion, and/or deletion in a protein or peptide sequence. An “amino acid substitution” or “substitution” refers to replacement of an amino acid at a particular position in a parent peptide or protein sequence with another amino acid. A substitution can be made to change an amino acid in the resulting protein in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein. The following are examples of various groupings of amino acids: 1) Amino acids with nonpolar R groups: Alanine, Valine, Leucine, Isoleucine, Proline, Phenylalanine, Tryptophan, Methionine; 2) Amino acids with uncharged polar R groups: Glycine, Serine, Threonine, Cysteine, Tyrosine, Asparagine, Glutamine; 3) Amino acids with charged polar R groups (negatively charged at pH 6.0): Aspartic acid, Glutamic acid; 4) Basic amino acids (positively charged at pH 6.0): Lysine, Arginine, Histidine (at pH 6.0). Another grouping may be those amino acids with phenyl groups: Phenylalanine, Tryptophan, and Tyrosine.

In some cases, the CD45 CAR or CD45 polypeptide can be produced using a vector in which the CAR open reading frame is followed by a T2A ribosome skip sequence and a truncated EGFR (EGFRt), which lacks the cytoplasmic signaling tail. In this arrangement, co-expression of EGFRt provides an inert, non-immunogenic surface marker that allows for accurate measurement of gene modified cells, and enables positive selection of gene-modified cells, as well as efficient cell tracking of the therapeutic T cells in vivo following adoptive transfer. Efficiently controlling proliferation to avoid cytokine storm and off-target toxicity is an important hurdle for the success of T cell immunotherapy. The EGFRt incorporated in the CD45 CAR lentiviral vector can act as suicide gene to ablate the CAR+ T cells in cases of treatment-related toxicity.

The CAR or polypeptide described herein can be produced by any means known in the art, though preferably it is produced using recombinant DNA techniques. Nucleic acids encoding the several regions of the chimeric receptor can be prepared and assembled into a complete coding sequence by standard techniques of molecular cloning known in the art (genomic library screening, overlapping PCR, primer-assisted ligation, site-directed mutagenesis, etc.) as is convenient. The resulting coding region is preferably inserted into an expression vector and used to transform a suitable expression host cell line, preferably a T lymphocyte, and most preferably an autologous T lymphocyte.

Various T cell subsets isolated from the patient can be transduced with a vector for CAR or polypeptide expression. Central memory T cells are one useful T cell subset. Central memory T cell can be isolated from peripheral blood mononuclear cells (PBMC) by selecting for CD45RO+/CD62L+ cells, using, for example, the CliniMACS® device to immunomagnetically select cells expressing the desired receptors. The cells enriched for central memory T cells can be activated with anti-CD3/CD28, transduced with, for example, a lentiviral vector that directs the expression of an CD45 CAR or CD45 polypeptide as well as a non-immunogenic surface marker for in vivo detection, ablation, and potential ex vivo selection. The activated/genetically modified CD45 central memory T cells can be expanded in vitro with IL-2/IL-15 and then cryopreserved. Additional methods of preparing CAR T cells can be found in PCT/US2016/043392.

Methods for preparing useful T cell populations are described in, for example, WO 2017/015490 and WO 2018/102761. In some cases, it may be useful to use natural killer (NK) cells, e.g., allogenic NK cells derived from peripheral blood or cord blood. In other cases, NK cells can be derived from human embryonic stem cells (hESCs) or induced pluripotent stem cells (iPSCs).

In some embodiments, described herein is a composition comprising the iPSC-derived CAR T cells or CAR NK cells. In some embodiments, a composition comprising iPSC-derived CAR T cells or CAR NK cells has enhanced therapeutic properties. In some embodiments, the iPSC-derived CAR T cells or CAR NK cells demonstrate enhanced functional activity including potent cytokine production, cytotoxicity and cytostatic inhibition of tumor growth, e.g. as activity that reduces the amount of tumor load.

The CAR can be transiently expressed in a T cell population by an mRNA encoding the CAR. The mRNA can be introduced into the T cells by electroporation (Wiesinger et al. 2019 Cancers (Basel) 11:1198).

In some embodiments, a composition comprising the CAR T cells comprise one or more of helper T cells, cytotoxic T cells, memory T cells, naïve T cells, regulatory T cells, natural killer T cells, or combinations thereof. In some embodiments, a composition comprising the CAR T cells comprise CD3+, CD5+, CD7+, and TCRαβ+. In some embodiments, a composition comprising the CAR T cells comprise CD8+ CAR T cells are CD8αβ T cells, which have strong cytotoxicity against tumor cells in an antigen specific manner and can potently secret cytokines such as IFNγ. In some embodiments, CAR T cells have predominant homogenous TCR phenotype. In some embodiments, a composition comprising the CAR T cells comprise CD3+CD5+CD7+TCRαβ+CD8αβ+, CD3+CD5+CD7+TCRαβ+CD4+, CD62L+CD45RA+ stem memory T cells, CD62L-CD45RA-CD45RO+ effector memory T cells and CD62L-CD45RA+ effector T cells, and combinations thereof.

In some embodiments, the fratricide effect is less than about 10%, 9%, 8%, 7%, 6%, 5%, 4%, 3%, 2%, or 1%.

In some embodiments, one or more genes are knocked out, knocked down, mutated, down regulated, or upregulated. In some embodiments, the one or more genes comprise the gene encoding CD45 (PTPRC). In some embodiments, CD45 (PTPRC) is knocked out. In some embodiments, CD45 (PTPRC) is down regulated. In some embodiments, genetic modification is achieved by methods described herein and those known in the art. In some embodiments, genetic modification methods comprise gene editing, homologous recombination, non-homologous recombination, RNA-mediated genetic modification, DNA-mediated genetic modification, zinc finger nucleases, meganucleases, TALEN, or CRISPR/CAS9. In some embodiments, the CRISPR/CAS9 system comprises a gRNA targeting a CD45 exon. In some embodiments, the CRISPR/CAS9 system comprises a gRNA targeting any one of CD45 exon #3, CD45 exon #8, CD45 exon #12, or CD45 exon #25. In some embodiments, the CRISPR/CAS9 system comprises a gRNA comprising or consisting of AUAUUAAUUCUUACCAGUGG (SEQ ID NO:37) or a variant thereof with 1, 2, 3, 4, or 5 nucleotide changes. In some embodiments, the CRISPR/CAS9 system comprises a gRNA comprising or consisting of ACUCCAUCUAAGCCAACAUG (SEQ ID NO:38) or a variant thereof with 1, 2, 3, 4, or 5 nucleotide changes. In some embodiments, the CRISPR/CAS9 system comprises a gRNA comprising or consisting of CUUCUACAAAAAAUAAUCUG (SEQ ID NO:39) or a variant thereof with 1, 2, 3, 4, or 5 nucleotide changes. In some embodiments, the CRISPR/CAS9 system comprises a gRNA comprising or consisting of GUGCUGGUGUUGGGCGC (SEQ ID NO:40) or a variant thereof with 1, 2, 3, 4, or 5 nucleotide changes. In some embodiments, the CRISPR/CAS9 system comprises a gRNA comprising or consisting of a sequence selected from the group consisting of: UUAUGAAAUGAUCUUUGAGG (SEQ ID NO: 41; exon #12); AAAAUAAUCUGAGGCUCUCC (SEQ ID NO: 42; exon #12); AUAGUAUGCAUGUCAAGUGU (SEQ ID NO: 43; exon #14); GGGCCAUUACGGUCCCUGGG (SEQ ID NO: 44; exon #14) or a variant of any of these with 1, 2, 3, 4, or 5 nucleotide changes.

In some embodiments, the methods described herein provide for controlling the persistence of CAR T cells and CAR NK cells via an inducible suicide switch or transient expression by mRNA CAR technology. In some embodiments, the CAR comprises an inducible “suicide switch” or transduction of the CAR is conducted for transient expression to effectively control the persistence of CAR T cells and CAR NK cells. In some embodiments, this mitigates the risk of hematopoietic toxicity and facilitates clinical application of the CD45-CAR T cells or CD45-CAR NK cells described herein. In some embodiments, the CAR constructs described herein comprise an inducible suicide switch. In some embodiments, the inducible suicide gene is any one of inducible Caspase 9 (iCasp9), EGFR (and/or tEGFR), herpes simplex virus tyrosine kinase (HSV-TK), or human thymidylate kinase (TMPK); other inducible suicide switches are known in the art. Without being bound by theory, cells expressing constructs comprising iCasp9 or TMPK, elimination is achieved through activation of the caspase 3 apoptotic pathway when a small molecule is administered. In some embodiments, the suicide switch is induced by an antibody, such as a clinically approved antibody, e.g., rituximab targeting CD20; cetuximab, panitumumab, zalutumumab, nimotuzumab, matuzumab, or any other antibody targeting EGFR. In some embodiments, the suicide switch is induced by a small molecule or drug, such as a specific chemical inducer of dimerization (CID); e.g., ganciclovir to target TMPK; ramiducid to target iCASp9; etc. Other ways to target or induce a suicide switch are known in the art. In some embodiments, the inducible suicide switch is N-terminal to an scFv or the inducible suicide switch is C-terminal to an scFv. In some embodiments, the inducible suicide switch and scFv are joined by a flexible linker or skip sequence as described herein (e.g., SEQ ID NO:60-61, or a variant hereof having 1, 2, 3, 4, 5, 6, 7, 8, 9, or 10 single amino acid substitutions). In some embodiments, the inducible suicide switch is C-terminal to a costimulatory domain or a CD3ζ domain. For example, a CAR can comprise an EGFR or tEGFR domain as described herein (e.g., SEQ ID NO:31) and targeted via an antibody (e.g., panitumumab).

(iCasp9-T2A-CD45 scFV)
SEQ ID NO: 60
MLEGVQVETISPGDGRTFPKRGQTCVVHYTGMLEDGKKVDSSRDRNKPFKFMLGK
QEVIRGWEEGVAQMSVGQRAKLTISPDYAYGATGHPGIIPPHATLVFDVELLKLESG
GGSGVDGFGDVGALESLRGNADLAYILSMEPCGHCLIINNVNFCRESGLRTRTGSNID
CEKLRRRFSSLHFMVEVKGDLTAKKMVLALLELARQDHGALDCCVVVILSHGCQAS
HLQFPGAVYGTDGCPVSVEKIVNIFNGTSCPSLGGKPKLFFIQACGGEQKDHGFEVAS
TSPEDESPGSNPEPDATPFQEGLRTFDQLDAISSLPTPSDIFVSYSTFPGFVSWRDPKSG
SWYVETLDDIFEQWAHSEDLQSLLLRVANAVSVKGIYKQMPGCFNFLRKKLFFKTE
GRGSLLTCGDVEENPGPQVQLVESGGGLVQPGGSLKLSCAASGFDFSRYWMSWVR
QAPGKGLEWIGEINPTSSTINFTPSLKDKVFISRDNAKNTLYLQMSKVRSEDTALYYC
ARGNYYRYGDAMDYWGQGTSVTVSKISGGGGSGGGGSGGGGSGGGGSGGGGSSDI
VLTQSPASLAVSLGQRATISCRASKSVSTSGYSYLHWYQQKPGQPPKLLIYLASNLES
GVPARFSGSGSGTDFTLNIHPVEEEDAATYYCQHSRELPFTFGSGTKLEIK
(CD45 scFV-T2A-iCasp9)
SEQ ID NO: 61
QVQLVESGGGLVQPGGSLKLSCAASGFDFSRYWMSWVRQAPGKGLEWIGEINPTSS
TINFTPSLKDKVFISRDNAKNTLYLQMSKVRSEDTALYYCARGNYYRYGDAMDYW
GQGTSVTVSKISGGGGSGGGGSGGGGSGGGGSGGGGSSDIVLTQSPASLAVSLGQRA
TISCRASKSVSTSGYSYLHWYQQKPGQPPKLLIYLASNLESGVPARFSGSGSGTDFTL
NIHPVEEEDAATYYCQHSRELPFTFGSGTKLEIKEGRGSLLTCGDVEENPGPMLEGVQ
VETISPGDGRTFPKRGQTCVVHYTGMLEDGKKVDSSRDRNKPFKFMLGKQEVIRGW
EEGVAQMSVGQRAKLTISPDYAYGATGHPGIIPPHATLVFDVELLKLESGGGSGVDG
FGDVGALESLRGNADLA YILSMEPCGHCLIINNVNFCRESGLRTRTGSNIDCEKLRRR
FSSLHFMVEVKGDLTAKKMVLALLELARQDHGALDCCVVVILSHGCQASHLQFPGA
VYGTDGCPVSVEKIVNIFNGTSCPSLGGKPKLFFIQACGGEQKDHGFEVASTSPEDES
PGSNPEPDATPFQEGLRTFDQLDAISSLPTPSDIFVSYSTFPGFVSWRDPKSGSWYVET
LDDIFEQWAHSEDLQSLLLRVANAVSVKGIYKQMPGCFNFLRKKLFFKT

In some embodiments, transient expression is induced by mRNA CAR technology. In some embodiments, the mRNA encodes a CD45 CAR, a CD45 CAR with an inducible suicide switch, and/or an mRNA encoding a CD45 CAR and an mRNA encoding a suicide switch (e.g., iCaspase9). Methods of administering mRNA to a population of T cells or NK cells are known in the art (e.g., transfecting via electroporation). In some embodiments, the population of T cells or population of NK cells transfected by an mRNA has been genetically altered as described herein (e.g., CD45 (PTPRC) has been knocked out, knocked down, mutated, down regulated, or upregulated). In some embodiments the mRNA comprises an 2bgUTR (beta globulin untranslated region; SEQ ID NO:55) and/or a polyA (pA) sequence; e.g., 100 pA, 110 pA, 120 pA, 130 pA, 140 pA, 150 pA (SEQ ID NO:56), etc. In some embodiments, the 2bgUTR and 150 pA increase the stability of an mRNA encoding a CAR (Modification of antigen-encoding RNA increases stability, translational efficacy, and T-cell stimulatory capacity of dendritic cells. Blood (2006) 108 (13): 4009-4017). In some embodiments, the mRNA comprises a promoter sequence; e.g., T7 (SEQ ID NO:57), T3, SP6, etc. An mRNA can encode any scFv or CAR described herein (SEQ ID NOs: 1, 29-31, 62-82); for example, an mRNA can comprise or consist of the sequence in FIG. 12B (SEQ ID NO:58) CD45scfv-IgG4(L235E, N297Q)-CD28tm-CD28gg-Zeta

SEQ ID NO: 55
cagctcgctttcttgctgtccaatttctattaaaggttcctttgttccct
aagtccaactactaaactgggggatattatgaagggccttgagcatctgg
attctgcctaataaaaaacatttattttcattgctgcgtcgagagctcgc
tttcttgctgtccaatttctattaaaggttcctttgttccctaagtccaa
ctactaaactgggggatattatgaagggccttgagcatctggattctgcc
taataaaaaacatttattttcattgctgcctcga
SEQ ID NO: 56
aaaaaaaaaaaaaaaaaaaaaaaaaaaaaaaaaaaaaaaaaaaaaaaaaa
aaaaaaaaaaaaaaaaaaaaaaaaaaaaaaaaaaaaaaaaaaaaaaaaaa
aaaaaaaaaaaaaaaaaaaaaaaaaaaaaaaaaaaaaaaaaaaaaaaaaa
SEQ ID NO: 57
taatacgactcactatagg

In some embodiments, described herein is a method of increasing survival of a subject having cancer comprising administering a composition comprising a CAR T cell or CAR NK cell described herein.

In some embodiments, described herein is a method of treating a cancer in a patient comprising administering a composition comprising a CAR T cell or CAR NK cell described herein.

In some embodiments, described herein is a method of reducing or ameliorating a symptom associated with a cancer in a patient comprising administering a composition comprising a CAR T cell or CAR NK cell described herein.

In some embodiments, a composition comprising CAR T cells or CAR NK cells described herein is administered locally or systemically. In some embodiments, a composition comprising CAR T cells or CAR NK cells described herein is administered by single or repeat dosing. In some embodiments, a composition comprising CAR T cells or CAR NK cells described herein is administered to a patient having a cancer, a pathogen infection, an autoimmune disorder, or undergoing allogeneic transplant.

In some embodiments, the cancer is selected from the group consisting of blood cancer, B cell leukemia, multiple myeloma, lymphoblastic leukemia (ALL), chronic lymphocytic leukemia, non-Hodgkin's lymphoma, ovarian cancer, prostate cancer, pancreatic cancer, lung cancer, breast cancer, and sarcoma, acute myeloid leukemia (AML).

Also provided herein is a method of enhancing T cell proliferation in T cells expressing a CAR comprising knocking out, knocking down, or mutating the PTPRC gene in the T cells thereby creating CD45 CAR T cells. In some embodiments, there is less than 25%, 20%, 15%, 10%, 9%, 8%, 7%, 6%, 5%, 4%, 3%, 2%, or 1% decrease in antigen-specific toxicity of the CD45− CAR T cells compared the CD45+ CAR T cells expressing the same CAR.

The materials, methods, and examples are illustrative only and not intended to be limiting. All publications, patent applications, patents, sequences, database entries, and other references mentioned herein are incorporated by reference in their entirety for any and all purposes.

Other features and advantages of the described compositions and methods will be apparent from the following detailed description and figures, and from the claims.

### DESCRIPTION OF DRAWINGS

FIGS. 1A-1B show a schematic depicting CD45 CAR constructs (A) and the annotated amino acid sequences of a CD45 CAR (SEQ ID NO.29; B).

FIGS. 2A-2D show generation of CD45 knockout CD45CAR T cells. FIG. 2A shows a timeline schematic to generate CD45 knockout CD45CAR T cells. To prevent the strong fratricide effect of WT CD45CAR T cells, CD45 (PTPRC) gene was knocked out on T cells before CD45CAR lentivirus transduction. CRISPR-cas9 RNP system were used to edit the CD45 genes with guide RNAs (gRNA) targeting different exons (E3, 8, 12, 25) of CD45. FIG. 2B shows the growth curves of CD45CAR T cells. FIG. 2C shows the expression profiles of CAR was evaluated by truncated EGFR staining. FIG. 2D shows the presence of CD45, CD4 and CD8 of the CAR+ T cells.

FIG. 3 shows CD45 expression profiles of hematopoietic malignant cells. Myeloid leukemia cell lines (KG1A, MV4-11, K562), T cell leukemia and lymphoma cell lines (Jurkat, CEM and Hut78), B cell leukemia and blast cell lines (TM-LCL, Raji and NALM6), multiple myeloma cell line MM.1S were stained with anti-CD45 antibody (BD55482, clone HI30) and analyzed by flow cytometry.

FIGS. 4A-4E show Cytotoxicity of CD45CAR T cells against different hematopoietic malignant cell lines. Indicated tumor cells were co-cultured with CAR T/T cells in series of Effector: Target (E:T) ratios for 4h (FIGS. 4A-4D) or 48h (FIG. 4E) and cytotoxic effect was measured by flow cytometry. (FIG. 4A) Cytotoxicity of CD45KO CD45CAR T cells generated by different gRNAs. 4h cytotoxicity of gRNA #3 generated CD45KO CD45 CAR T cells against myeloid leukemia cell line KG1A (FIG. 4B), B-ALL line Raji (FIG. 4C), and blast crisis CML cell line KCL22M with T315I BCR-ABL mutation (FIG. 4D). 48h cytotoxicity effect against AML cell line MV4-11, multiple myeloma cell line MM.1S, and B lymphoblast cell line TM-LCL (FIG. 4E).

FIGS. 5A-5C show myeloid and lymphoid depletion effect of CD45KO CD45CAR T cells against CD45+ healthy hematopoietic cells. PBMCs from heathy donor were co-cultured with CFSE labeled CAR T (FIGS. 5A and 5B) or T cells (FIG. 5C) in series of Effector: Target (E:T) ratios for 4h (FIG. 5A) or 24h (FIGS. 5B and 5C) and cytotoxic effect was analyzed by flow cytometry in combination with myeloid cells (CD11b+), T (CD3+) and B (CD19+) cell markers.

FIG. 6 shows degranulation activity and IFNγ production of CD45CAR T cells against CD45+ AML KG1A cells. CD45KO CD45 CAR T or mock T cells were cocultured with KG1A cells with Effector: Target (E:T) ratios 1:1 for 5h and the degranulation activity was indicated by CD107a staining together with IFNγ intracellular staining.

FIGS. 7A-7D shows myeloid depletion effect of CD45CAR T cells in an AML mouse model. (FIG. 7A) NSG mice were engrafted with 1 million MV4-11.eGFP.ffluc cells by i.v. injection. 5 days later, tumor cells were measured by noninvasive optical bio-photonic imaging using a Xenogen IVIS system and the mice were treated with 2 million mock T cells or CD45 CAR T cells (CD45KO). The tumor growth was monitored every week by imaging (FIGS. 7B-7C). (FIG. 7D) The health status and survival was tracked, and survival curve was monitored.

FIGS. 8A-8E shows CD45 Knockout enhance CD19CAR T cells proliferation and preserve the functionality. (FIG. 8A) Surface marker profiles of wild type (WT) and CD45KO CD19-CAR T cells. (FIG. 8B) Degranulation activity and IFNγ production of WT and CD45KO CD19-CAR T cells with 4h-coculture with CD19 negative KG1A cells and CD19 positive cells (TM-LCL, NALM6 and Raji). The labeled number is the percentage of positive populations. (FIG. 8C) Proliferation of FACS sorted T cells and CD19-CART cells with and without CD45 knockout. The cells were cultured for 26 days. (FIG. 8D) Cytotoxicity of WT and CD45KO CD19-CAR T cells against CD19 negative KG1A cells and CD19 positive NALM6 cells with effector: target (E:T) ratio of 5:1 for 4h. (FIG. 8E) Cytotoxicity of WT and CD45KO CD19-CAR T cells against CD19 positive NALM6 cells and CD19KO NALM6 cells with series of effector: target (E:T) ratios for 4h.

FIG. 9 shows antigen specific anti-tumor and myeloid-/lymphoid-ablation effect of CD45-CAR T cells. Luciferase-based Cytotoxicity Assay (LCA) of CD45-CAR T cells against different target cells with 48-hour co-culture in different Effector (E): Target (T) ratios. All the target cells were engineered with firefly luciferase (ffLuc+).

FIGS. 10A-10C show induced cell depletion effect of rimiducid on iCasp9-CD45-CAR lentivirus transduced cells. FIG. 10A is a schematic showing an iCasp9-CD45-CAR construct containing iCaspase9 as safety switch. The order of iCaspase 9 and CD45CAR components can be switched (e.g., promoter-CD45CAR-T2A-iCaspase9-etc). FIG. 10B shows flow cytometry results of HT1080 cells transduced by iCasp9-CD45-CAR lentivirus.

FIG. 10C shows a bar graph quantifying relative survival cell number in an experiment where the same number of transduced HT1080 cells as in FIG. 10B were seeded in 24 well plate and treated with 100 μM ramiducid for 24h normalized and compared to non-treated group.

FIGS. 11A-11B show CD45 protein expression profile after PTPRC (CD45) gene knockout by CRISPR/Cas9 gene editing. Data show flow cytometry profiles (FIG. 11A) and quantified MFI (FIG. 11B) of CD45 expression on wild type and CD45 knock out T cells. hCD45gRNA #3_E12, CUUCUACAAAAAAUAAUCUG (SEQ ID NO: 39) was used for this experiment.

FIGS. 12A-12B show a schematic (FIG. 12A) and sequence (FIG. 12B) of the cDNA of a CD45 mRNA CAR construct with a T7 promoter and stabilization sequences (SEQ ID NO:58).

FIGS. 13A-13B show a schematic (FIG. 13A) and amino acid sequence (FIG. 13B) of the iCaspase 9 mRNA as safety switch construct (SEQ ID NO:59).

FIGS. 14A-14C show characteristics of CD45KO CD45CAR T cells generated by mRNA transduction. FIG. 14A shows the CD45 expression profile on wild type T cells, CD45KO T cells, and CD45KO CD45CAR T cells. FIG. 14B shows CAR expression profile of CD45KO CD45CAR T cells 24h after mRNA electroporation. FIG. 14C shows cytotoxicity of CD45KO CD45CAR T cells generated by mRNA transduction against tumor cells. Raji cells were co-cultured with CAR T cells for 24h.

FIG. 15 shows mRNA electroporated T cells preserve expression for 2 weeks. T cells cultured for 7 days were transduced via electroporation with GFP mRNA in a dose of 2.5 ug/million and the expression level of GFP expression was tracked by flow cytometry.

FIGS. 16A-16G. Amino acid sequences of CD45 scFv CAR. (FIG. 16A) CD45 scFv CAR with IgG4(HL-CH3) spacer, CD28 transmembrane domain, CD28 co-stimulatory domain, and CD3 zeta domain (SEQ ID NO:62), without signal sequence (SEQ ID NO:63), and with tEGFR (SEQ ID NO:64). (FIG. 16B) CD45 scFv CAR with IgG4(S228P, L235E,N297Q) spacer, NKG2D transmembrane domain, 2B4 co-stimulatory domain, and CD3 zeta domain (SEQ ID NO:65), without signal sequence (SEQ ID NO:66), and with tEGFR (SEQ ID NO:67). (FIG. 16C) CD45 scFv CAR with IgG4(HL-CH3) spacer, NKG2D transmembrane domain, 2B4 co-stimulatory domain, and CD3 zeta domain (SEQ ID NO:68), without signal sequence (SEQ ID NO:69) and with tEGFR (SEQ ID NO:70). (FIG. 16D) CD45 scFv CAR with CD8h spacer, NKG2D transmembrane domain, 2B4 co-stimulatory domain, and CD3 zeta domain (SEQ ID NO:71), without signal sequence (SEQ ID NO:72), and with tEGFR (SEQ ID NO:73). (FIG. 16E) CD45 scFv CAR with CD8h spacer, CD8 transmembrane domain, 41BB co-stimulatory domain, and CD3 zeta domain (SEQ ID NO:74), without signal sequence (SEQ ID NO:75), and with tEGFR (SEQ ID NO:76). (FIG. 16F) CD45 scFv CAR with IgG4(HL-CH3) spacer, CD4 transmembrane domain, 41BB co-stimulatory domain, and CD3 zeta domain (SEQ ID NO:77), without signal sequence (SEQ ID NO:78), and with tEGFR (SEQ ID NO:79). (FIG. 16G) CD45 scFv CAR IgG4(S228P, L235E,N297Q) spacer, CD4 transmembrane domain, 41BB co-stimulatory domain, and CD3 zeta domain (SEQ ID NO:80), without signal sequence (SEQ ID NO:81), and with tEGFR (SEQ ID NO:82).

### DETAILED DESCRIPTION

In this disclosure, the generation and anti-tumor efficacy of CAR with an anti-CD45 scFv antigen-binding domain are described, inter alia.

Hematopoietic transplantation has been proven effective to treat a wide array of malignant and non-malignant hematological diseases. The preparative regimen, however, routinely entails aggressive and genotoxic treatment with whole body irradiation and/or chemotherapy, which can introduce severe and even life-threatening complications. Ablation of recipient bone marrow cells, including myeloid cells and HSCs, is a requirement of these conditioning regimens in order allow successful engraftment of the composite donor HSCs. Alternative conditioning approaches for bone marrow transplantation (BMT) with less toxic side-effects are desirable. CD45 is a hematopoietic lineage specific marker. Precise hematopoietic cells targeting may be achieved by the application of CD45 targeting chimeric antigen receptor (CAR) T or CAR NK cells for hematological cell transplantation conditioning. CD45 is also a widely expressed surface marker of different types of hematological malignancies including AML, B-ALL, T-ALL, and CML. CD45CAR T cells or CAR NK cells can also be used to treat CD45 positive hematological malignancies. To prevent the fratricide effect of CD45 CAR T or CAR NK cells, the CD45 (PTPRC) gene can be knockout out or mutated by gene editing technologies.

The present disclosure relates to novel chimeric antigen receptors (CARs) and applications thereof. CARs are able to redirect immune cell specificity and reactivity toward a selected target through exploiting the ligand-binding domain properties. In particular, the present disclosure relates to a Chimeric Antigen Receptor with extracellular scFv domain of a CD45 monoclonal antibody (e.g., BC8 clone). The present disclosure also relates to polynucleotides, vectors encoding said CAR and genetically modified immune cells expressing said CAR at their surface. The present disclosure also relates to methods to gene edit immune cells by knockout, knockdown or mutant CD45 gene along with co-expressing CD45CAR to produce fratricide resistant CD45CAR T cells or CD45CAR NK cells. The present disclosure is particularly useful for myeloid ablation, hematological cell transplantation conditioning and for the treatment of CD45 positive hematopoietic malignancies such as myeloid leukemia, T cell leukemia/lymphomas, and the like.

### Examples

The CD45 CAR and their use is further described in the following examples, which do not limit the scope the claims.

Materials and Methods

The following materials and methods were used in the Examples set forth herein.

Cell Lines Myeloid leukemia cell lines (KG1A, MV4-11, K562), T cell leukemia and lymphoma cell lines (Jurkat, CEM, and Hut78), B cell leukemia and blast cell lines (TM-LCL, Raji and NALM6), multiple myeloma cell line (MM.1S) were cultured in RPMI-1640 (Lonza) containing 10% fetal bovine serum (FBS, Hyclone) (complete RPMI). The 293T and HT1080 cell lines were cultured in Dulbecco's Modified Eagles Medium (DMEM, Life Technologies) containing 10% FBS, 1×AA, 25 mM HEPES (Irvine Scientific), and 2 mM L-Glutamine (Fisher Scientific) (complete DMEM). All cells were cultured at 37° C. with 5% CO₂. HUT78 cells were cultured in IMDM (Iscove's Modified Dulbecco's Medium; Fisher Scientific) with 20% FBS.

DNA Constructs and Lentivirus Production

Tumor cells were engineered to express enhanced green fluorescent protein and firefly luciferase (eGFP/ffluc) by transduction with epHIV7 lentivirus carrying the eGFP/ffluc fusion under the control of the EF1α promoter as described previously (Lenalidomide Enhances the Function of CS1 Chimeric Antigen Receptor-Redirected T Cells Against Multiple Myeloma (Wang et al). Clinical Cancer Research 2018).

Research grade lentivirus was generated using a modified polyethylenimine (PEI) mediated transfection method (Optimization of lentiviral vector production using polyethylenimine-mediated transfection. Yong Tang, et al. Oncology Letters. 2015). Briefly, 293T cells were transfected with packaging plasmid and CAR lentiviral backbone plasmid using a modified PEI method. Viral supernatants were collected after 3 to 4 days. Supernatants were concentrated via high-speed centrifugation and lentiviral pellets were resuspended in phosphate-buffered saline (PBS)-lactose solution (4 g lactose per 100 mL PBS), aliquoted and stored at −80° C. Lentiviral titers were quantified using HT1080 cells based on EGFRt expression.

(CD45 CAR with signal sequence)
SEQ ID NO: 29
MLLLVTSLLLCELPHPAFLLIPQVQLVESGGGLVQPGGSLKLSCAASGFDFSRYWMS
WVRQAPGKGLEWIGEINPTSSTINFTPSLKDKVFISRDNAKNTLYLQMSKVRSEDTAL
YYCARGNYYRYGDAMDYWGQGTSVTVSKISGGGGSGGGGSGGGGSGGGGSGGGG
SSDIVLTQSPASLAVSLGQRATISCRASKSVSTSGYSYLHWYQQKPGQPPKLLIYLAS
NLESGVPARFSGSGSGTDFTLNIHPVEEEDAATYYCQHSRELPFTFGSGTKLEIKESKY
GPPCPPCPAPEFEGGPSVFLFPPKPKDTLMISRTPEVTCVVVDVSQEDPEVQFNWYVD
GVEVHNAKTKPREEQFQSTYRVVSVLTVLHQDWLNGKEYKCKVSNKGLPSSIEKTIS
KAKGQPREPQVYTLPPSQEEMTKNQVSLTCLVKGFYPSDIAVEWESNGQPENNYKT
TPPVLDSDGSFFLYSRLTVDKSRWQEGNVFSCSVMHEALHNHYTQKSLSLSLGKMF
WVLVVVGGVLACYSLLVTVAFIIFWVRSKRSRGGHSDYMNMTPRRPGPTRKHYQP
YAPPRDFAAYRSGGGRVKFSRSADAPAYQQGQNQLYNELNLGRREEYDVLDKRRG
RDPEMGGKPRRKNPQEGLYNELQKDKMAEAYSEIGMKGERRRGKGHDGLYQGLST
ATKDTYDALHMQALPPR
(CD45 CAR)
SEQ ID NO: 30
QVQLVESGGGLVQPGGSLKLSCAASGFDFSRYWMSWVRQAPGKGLEWIGEINPTSS
TINFTPSLKDKVFISRDNAKNTLYLQMSKVRSEDTALYYCARGNYYRYGDAMDYW
GQGTSVTVSKISGGGGSGGGGSGGGGSGGGGSGGGGSSDIVLTQSPASLAVSLGQRA
TISCRASKSVSTSGYSYLHWYQQKPGQPPKLLIYLASNLESGVPARFSGSGSGTDFTL
NIHPVEEEDAATYYCQHSRELPFTFGSGTKLEIKESKYGPPCPPCPAPEFEGGPSVFLF
PPKPKDTLMISRTPEVTCVVVDVSQEDPEVQFNWYVDGVEVHNAKTKPREEQFQST
YRVVSVLTVLHQDWLNGKEYKCKVSNKGLPSSIEKTISKAKGQPREPQVYTLPPSQE
EMTKNQVSLTCLVKGFYPSDIAVEWESNGQPENNYKTTPPVLDSDGSFFLYSRLTVD
KSRWQEGNVFSCSVMHEALHNHYTQKSLSLSLGKMFWVLVVVGGVLACYSLLVTV
AFIIFWVRSKRSRGGHSDYMNMTPRRPGPTRKHYQPYAPPRDFAAYRSGGGRVKFS
RSADAPAYQQGQNQLYNELNLGRREEYDVLDKRRGRDPEMGGKPRRKNPQEGLYN
ELQKDKMAEAYSEIGMKGERRRGKGHDGLYQGLSTATKDTYDALHMQALPPR
(CD45 CAR with signal sequence and EGFRt)
SEQ ID NO: 31
MLLLVTSLLLCELPHPAFLLIPQVQLVESGGGLVQPGGSLKLSCAASGFDFSRYWMS
WVRQAPGKGLEWIGEINPTSSTINFTPSLKDKVFISRDNAKNTLYLQMSKVRSEDTAL
YYCARGNYYRYGDAMDYWGQGTSVTVSKISGGGGSGGGGSGGGGSGGGGSGGGG
SSDIVLTQSPASLAVSLGQRATISCRASKSVSTSGYSYLHWYQQKPGQPPKLLIYLAS
NLESGVPARFSGSGSGTDFTLNIHPVEEEDAATYYCQHSRELPFTFGSGTKLEIKESKY
GPPCPPCPAPEFEGGPSVFLFPPKPKDTLMISRTPEVTCVVVDVSQEDPEVQFNWYVD
GVEVHNAKTKPREEQFQSTYRVVSVLTVLHQDWLNGKEYKCKVSNKGLPSSIEKTIS
KAKGQPREPQVYTLPPSQEEMTKNQVSLTCLVKGFYPSDIAVEWESNGQPENNYKT
TPPVLDSDGSFFLYSRLTVDKSRWQEGNVFSCSVMHEALHNHYTQKSLSLSLGKMF
WVLVVVGGVLACYSLLVTVAFIIFWVRSKRSRGGHSDYMNMTPRRPGPTRKHYQP
YAPPRDFAAYRSGGGRVKFSRSADAPAYQQGQNQLYNELNLGRREEYDVLDKRRG
RDPEMGGKPRRKNPQEGLYNELQKDKMAEAYSEIGMKGERRRGKGHDGLYQGLST
ATKDTYDALHMQALPPRLEGGGEGRGSLLTCGDVEENPGPRMLLLVTSLLLCELPHP
AFLLIPRKVCNGIGIGEFKDSLSINATNIKHFKNCTSISGDLHILPVAFRGDSFTHTPPLD
PQELDILKTVKEITGFLLIQAWPENRTDLHAFENLEIIRGRTKQHGQFSLAVVSLNITSL
GLRSLKEISDGDVIISGNKNLCYANTINWKKLFGTSGQKTKIISNRGENSCKATGQVC
HALCSPEGCWGPEPRDCVSCRNVSRGRECVDKCNLLEGEPREFVENSECIQCHPECL
PQAMNITCTGRGPDNCIQCAHYIDGPHCVKTCPAGVMGENNTLVWKYADAGHVCH
LCHPNCTYGCTGPGLEGCPTNGPKIPSIATGMVGALLLLLVVALGIGLFMT
(CD45 scFv-IgG4(HL-CH3)-CD28 TM-CD28GG-CD3ζ with signal)
SEQ ID NO: 62
MLLLVTSLLLCELPHPAFLLIPQVQLVESGGGLVQPGGSLKLSCAASGFDFSRYWMS
WVRQAPGKGLEWIGEINPTSSTINFTPSLKDKVFISRDNAKNTLYLQMSKVRSEDTAL
YYCARGNYYRYGDAMDYWGQGTSVTVSKISGGGGSGGGGSGGGGSGGGGSGGGG
SSDIVLTQSPASLAVSLGQRATISCRASKSVSTSGYSYLHWYQQKPGQPPKLLIYLAS
NLESGVPARFSGSGSGTDFTLNIHPVEEEDAATYYCQHSRELPFTFGSGTKLEIKESKY
GPPCPPCPGGGSSGGGSGGQPREPQVYTLPPSQEEMTKNQVSLTCLVKGFYPSDIAVE
WESNGQPENNYKTTPPVLDSDGSFFLYSRLTVDKSRWQEGNVFSCSVMHEALHNHY
TQKSLSLSLGKMFWVLVVVGGVLACYSLLVTVAFIIFWVRSKRSRGGHSDYMNMTP
RRPGPTRKHYQPYAPPRDFAAYRSGGGRVKFSRSADAPAYQQGQNQLYNELNLGRR
EEYDVLDKRRGRDPEMGGKPRRKNPQEGLYNELQKDKMAEAYSEIGMKGERRRGK
GHDGLYQGLSTATKDTYDALHMQALPPR
(CD45 scFv-IgG4(HL-CH3)-CD28 TM-CD28GG-CD39)
SEQ ID NO: 63
QVQLVESGGGLVQPGGSLKLSCAASGFDFSRYWMSWVRQAPGKGLEWIGEINPTSS
TINFTPSLKDKVFISRDNAKNTLYLQMSKVRSEDTALYYCARGNYYRYGDAMDYW
GQGTSVTVSKISGGGGSGGGGSGGGGSGGGGSGGGGSSDIVLTQSPASLAVSLGQRA
TISCRASKSVSTSGYSYLHWYQQKPGQPPKLLIYLASNLESGVPARFSGSGSGTDFTL
NIHPVEEEDAATYYCQHSRELPFTFGSGTKLEIKESKYGPPCPPCPGGGSSGGGSGGQ
PREPQVYTLPPSQEEMTKNQVSLTCLVKGFYPSDIAVEWESNGQPENNYKTTPPVLD
SDGSFFLYSRLTVDKSRWQEGNVFSCSVMHEALHNHYTQKSLSLSLGKMFWVLVV
VGGVLACYSLLVTVAFIIFWVRSKRSRGGHSDYMNMTPRRPGPTRKHYQPYAPPRD
FAAYRSGGGRVKFSRSADAPAYQQGQNQLYNELNLGRREEYDVLDKRRGRDPEMG
GKPRRKNPQEGLYNELQKDKMAEAYSEIGMKGERRRGKGHDGLYQGLSTATKDTY
DALHMQALPPR
(CD45 scFv-IgG4(HL-CH3)-CD28 TM-CD28GG-CD3ζ with
signal and EGFRt)
SEQ ID NO: 64
MLLLVTSLLLCELPHPAFLLIPQVQLVESGGGLVQPGGSLKLSCAASGFDFSRYWMS
WVRQAPGKGLEWIGEINPTSSTINFTPSLKDKVFISRDNAKNTLYLQMSKVRSEDTAL
YYCARGNYYRYGDAMDYWGQGTSVTVSKISGGGGSGGGGSGGGGSGGGGSGGGG
SSDIVLTQSPASLAVSLGQRATISCRASKSVSTSGYSYLHWYQQKPGQPPKLLIYLAS
NLESGVPARFSGSGSGTDFTLNIHPVEEEDAATYYCQHSRELPFTFGSGTKLEIKESKY
GPPCPPCPGGGSSGGGSGGQPREPQVYTLPPSQEEMTKNQVSLTCLVKGFYPSDIAVE
WESNGQPENNYKTTPPVLDSDGSFFLYSRLTVDKSRWQEGNVFSCSVMHEALHNHY
TQKSLSLSLGKMFWVLVVVGGVLACYSLLVTVAFIIFWVRSKRSRGGHSDYMNMTP
RRPGPTRKHYQPYAPPRDFAAYRSGGGRVKFSRSADAPAYQQGQNQLYNELNLGRR
EEYDVLDKRRGRDPEMGGKPRRKNPQEGLYNELQKDKMAEAYSEIGMKGERRRGK
GHDGLYQGLSTATKDTYDALHMQALPPRLEGGGEGRGSLLTCGDVEENPGPRMLLL
VTSLLLCELPHPAFLLIPRKVCNGIGIGEFKDSLSINATNIKHFKNCTSISGDLHILPVAF
RGDSFTHTPPLDPQELDILKTVKEITGFLLIQAWPENRTDLHAFENLEIIRGRTKQHGQ
FSLAVVSLNITSLGLRSLKEISDGDVIISGNKNLCYANTINWKKLFGTSGQKTKIISNR
GENSCKATGQVCHALCSPEGCWGPEPRDCVSCRNVSRGRECVDKCNLLEGEPREFV
ENSECIQCHPECLPQAMNITCTGRGPDNCIQCAHYIDGPHCVKTCPAGVMGENNTLV
WKYADAGHVCHLCHPNCTYGCTGPGLEGCPTNGPKIPSIATGMVGALLLLLVVALG
IGLFMT
(CD45 scFv-IgG4(S228P, L235E, N297Q)-NKG2D TM-2B4-CD3ζ
with signal)
SEQ ID NO: 65
MLLLVTSLLLCELPHPAFLLIPQVQLVESGGGLVQPGGSLKLSCAASGFDFSRYWMS
WVRQAPGKGLEWIGEINPTSSTINFTPSLKDKVFISRDNAKNTLYLQMSKVRSEDTAL
YYCARGNYYRYGDAMDYWGQGTSVTVSKISGGGGSGGGGSGGGGSGGGGSGGGG
SSDIVLTQSPASLAVSLGQRATISCRASKSVSTSGYSYLHWYQQKPGQPPKLLIYLAS
NLESGVPARFSGSGSGTDFTLNIHPVEEEDAATYYCQHSRELPFTFGSGTKLEIKESKY
GPPCPPCPAPEFEGGPSVFLFPPKPKDTLMISRTPEVTCVVVDVSQEDPEVQFNWYVD
GVEVHNAKTKPREEQFQSTYRVVSVLTVLHQDWLNGKEYKCKVSNKGLPSSIEKTIS
KAKGQPREPQVYTLPPSQEEMTKNQVSLTCLVKGFYPSDIAVEWESNGQPENNYKT
TPPVLDSDGSFFLYSRLTVDKSRWQEGNVFSCSVMHEALHNHYTQKSLSLSLGKPFF
FCCFIAVAMGIRFIIMVTWRRKRKEKQSETSPKEFLTIYEDVKDLKTRRNHEQEQTFP
GGGSTIYSMIQSQSSAPTSQEPAYTLYSLIQPSRKSGSRKRNHSPSFNSTIYEVIGKSQP
KAQNPARLSRKELENFDVYSGGGRVKFSRSADAPAYQQGQNQLYNELNLGRREEY
DVLDKRRGRDPEMGGKPRRKNPQEGLYNELQKDKMAEAYSEIGMKGERRRGKGH
DGLYQGLSTATKDTYDALHMQALPPR
(CD45 scFv-IgG4(S228P, L235E, N297Q)-NKG2D TM-2B4-CD3ζ)
SEQ ID NO: 66
QVQLVESGGGLVQPGGSLKLSCAASGFDFSRYWMSWVRQAPGKGLEWIGEINPTSS
TINFTPSLKDKVFISRDNAKNTLYLQMSKVRSEDTALYYCARGNYYRYGDAMDYW
GQGTSVTVSKISGGGGSGGGGSGGGGSGGGGSGGGGSSDIVLTQSPASLAVSLGQRA
TISCRASKSVSTSGYSYLHWYQQKPGQPPKLLIYLASNLESGVPARFSGSGSGTDFTL
NIHPVEEEDAATYYCQHSRELPFTFGSGTKLEIKESKYGPPCPPCPAPEFEGGPSVFLF
PPKPKDTLMISRTPEVTCVVVDVSQEDPEVQFNWYVDGVEVHNAKTKPREEQFQST
YRVVSVLTVLHQDWLNGKEYKCKVSNKGLPSSIEKTISKAKGQPREPQVYTLPPSQE
EMTKNQVSLTCLVKGFYPSDIAVEWESNGQPENNYKTTPPVLDSDGSFFLYSRLTVD
KSRWQEGNVFSCSVMHEALHNHYTQKSLSLSLGKPFFFCCFIAVAMGIRFIIMVTWR
RKRKEKQSETSPKEFLTIYEDVKDLKTRRNHEQEQTFPGGGSTIYSMIQSQSSAPTSQE
PAYTLYSLIQPSRKSGSRKRNHSPSFNSTIYEVIGKSQPKAQNPARLSRKELENFDVYS
GGGRVKFSRSADAPAYQQGQNQLYNELNLGRREEYDVLDKRRGRDPEMGGKPRRK
NPQEGLYNELQKDKMAEAYSEIGMKGERRRGKGHDGLYQGLSTATKDTYDALHM
QALPPR
(CD45 scFv-IgG4(S228P, L235E, N297Q)-NKG2D TM-2B4-CD3ζ with
signal and EGFRt)
SEQ ID NO: 67
MLLLVTSLLLCELPHPAFLLIPQVQLVESGGGLVQPGGSLKLSCAASGFDFSRYWMS
WVRQAPGKGLEWIGEINPTSSTINFTPSLKDKVFISRDNAKNTLYLQMSKVRSEDTAL
YYCARGNYYRYGDAMDYWGQGTSVTVSKISGGGGSGGGGSGGGGSGGGGSGGGG
SSDIVLTQSPASLAVSLGQRATISCRASKSVSTSGYSYLHWYQQKPGQPPKLLIYLAS
GPPCPPCPAPEFEGGPSVFLFPPKPKDTLMISRTPEVTCVVVDVSQEDPEVQFNWYVD
GVEVHNAKTKPREEQFQSTYRVVSVLTVLHQDWLNGKEYKCKVSNKGLPSSIEKTIS
KAKGQPREPQVYTLPPSQEEMTKNQVSLTCLVKGFYPSDIAVEWESNGQPENNYKT
TPPVLDSDGSFFLYSRLTVDKSRWQEGNVFSCSVMHEALHNHYTQKSLSLSLGKPFF
FCCFIA VAMGIRFIIMVTWRRKRKEKQSETSPKEFLTIYEDVKDLKTRRNHEQEQTFP
GGGSTIYSMIQSQSSAPTSQEPAYTLYSLIQPSRKSGSRKRNHSPSFNSTIYEVIGKSQP
KAQNPARLSRKELENFDVYSGGGRVKFSRSADAPAYQQGQNQLYNELNLGRREEY
DVLDKRRGRDPEMGGKPRRKNPQEGLYNELQKDKMAEAYSEIGMKGERRRGKGH
DGLYQGLSTATKDTYDALHMQALPPRLEGGGEGRGSLLTCGDVEENPGPRMLLLVT
SLLLCELPHPAFLLIPRKVCNGIGIGEFKDSLSINATNIKHFKNCTSISGDLHILPVAFRG
DSFTHTPPLDPQELDILKTVKEITGFLLIQAWPENRTDLHAFENLEIIRGRTKQHGQFS
LAVVSLNITSLGLRSLKEISDGDVIISGNKNLCYANTINWKKLFGTSGQKTKIISNRGE
NSCKATGQVCHALCSPEGCWGPEPRDCVSCRNVSRGRECVDKCNLLEGEPREFVEN
SECIQCHPECLPQAMNITCTGRGPDNCIQCAHYIDGPHCVKTCPAGVMGENNTLVW
KYADAGHVCHLCHPNCTYGCTGPGLEGCPTNGPKIPSIATGMVGALLLLLVVALGIG
LFMT
(CD45 scFv-IgG4(HL-CH3)-NKG2D TM-2B4-CD3ζ with signal)
SEQ ID NO: 68
MLLLVTSLLLCELPHPAFLLIPQVQLVESGGGLVQPGGSLKLSCAASGFDFSRYWMS
WVRQAPGKGLEWIGEINPTSSTINFTPSLKDKVFISRDNAKNTLYLQMSKVRSEDTAL
YYCARGNYYRYGDAMDYWGQGTSVTVSKISGGGGSGGGGSGGGGSGGGGSGGGG
SSDIVLTQSPASLAVSLGQRATISCRASKSVSTSGYSYLHWYQQKPGQPPKLLIYLAS
NLESGVPARFSGSGSGTDFTLNIHPVEEEDAATYYCQHSRELPFTFGSGTKLEIKESKY
GPPCPPCPGGGSSGGGSGGQPREPQVYTLPPSQEEMTKNQVSLTCLVKGFYPSDIAVE
WESNGQPENNYKTTPPVLDSDGSFFLYSRLTVDKSRWQEGNVFSCSVMHEALHNHY
TQKSLSLSLGKPFFFCCFIAVAMGIRFIIMVTWRRKRKEKQSETSPKEFLTIYEDVKDL
KTRRNHEQEQTFPGGGSTIYSMIQSQSSAPTSQEPAYTLYSLIQPSRKSGSRKRNHSPS
FNSTIYEVIGKSQPKAQNPARLSRKELENFDVYSGGGRVKFSRSADAPAYQQGQNQL
YNELNLGRREEYDVLDKRRGRDPEMGGKPRRKNPQEGLYNELQKDKMAEAYSEIG
MKGERRRGKGHDGLYQGLSTATKDTYDALHMQALPPR
(CD45 scFv-IgG4(HL-CH3)-NKG2D TM-2B4-CD3ζ)
SEQ ID NO: 69
QVQLVESGGGLVQPGGSLKLSCAASGFDFSRYWMSWVRQAPGKGLEWIGEINPTSS
TINFTPSLKDKVFISRDNAKNTLYLQMSKVRSEDTALYYCARGNYYRYGDAMDYW
GQGTSVTVSKISGGGGSGGGGSGGGGSGGGGSGGGGSSDIVLTQSPASLAVSLGQRA
TISCRASKSVSTSGYSYLHWYQQKPGQPPKLLIYLASNLESGVPARFSGSGSGTDFTL
NIHPVEEEDAATYYCQHSRELPFTFGSGTKLEIKESKYGPPCPPCPGGGSSGGGSGGQ
PREPQVYTLPPSQEEMTKNQVSLTCLVKGFYPSDIAVEWESNGQPENNYKTTPPVLD
SDGSFFLYSRLTVDKSRWQEGNVFSCSVMHEALHNHYTQKSLSLSLGKPFFFCCFIA
VAMGIRFIIMVTWRRKRKEKQSETSPKEFLTIYEDVKDLKTRRNHEQEQTFPGGGSTI
YSMIQSQSSAPTSQEPAYTLYSLIQPSRKSGSRKRNHSPSFNSTIYEVIGKSQPKAQNP
ARLSRKELENFDVYSGGGRVKFSRSADAPAYQQGQNQLYNELNLGRREEYDVLDK
RRGRDPEMGGKPRRKNPQEGLYNELQKDKMAEAYSEIGMKGERRRGKGHDGLYQ
GLSTATKDTYDALHMQALPPR
(CD45 scFv-IgG4(HL-CH3)-NKG2D TM-2B4-CD3ζ with signal
and EGFRt)
SEQ ID NO: 70
MLLLVTSLLLCELPHPAFLLIPQVQLVESGGGLVQPGGSLKLSCAASGFDFSRYWMS
WVRQAPGKGLEWIGEINPTSSTINFTPSLKDKVFISRDNAKNTLYLQMSKVRSEDTAL
YYCARGNYYRYGDAMDYWGQGTSVTVSKISGGGGSGGGGSGGGGSGGGGSGGGG
SSDIVLTQSPASLAVSLGQRATISCRASKSVSTSGYSYLHWYQQKPGQPPKLLIYLAS
NLESGVPARFSGSGSGTDFTLNIHPVEEEDAATYYCQHSRELPFTFGSGTKLEIKESKY
GPPCPPCPGGGSSGGGSGGQPREPQVYTLPPSQEEMTKNQVSLTCLVKGFYPSDIAVE
WESNGQPENNYKTTPPVLDSDGSFFLYSRLTVDKSRWQEGNVFSCSVMHEALHNHY
TQKSLSLSLGKPFFFCCFIAVAMGIRFIIMVTWRRKRKEKQSETSPKEFLTIYEDVKDL
KTRRNHEQEQTFPGGGSTIYSMIQSQSSAPTSQEPAYTLYSLIQPSRKSGSRKRNHSPS
FNSTIYEVIGKSQPKAQNPARLSRKELENFDVYSGGGRVKFSRSADAPAYQQGQNQL
YNELNLGRREEYDVLDKRRGRDPEMGGKPRRKNPQEGLYNELQKDKMAEAYSEIG
MKGERRRGKGHDGLYQGLSTATKDTYDALHMQALPPRLEGGGEGRGSLLTCGDVE
ENPGPRMLLLVTSLLLCELPHPAFLLIPRKVCNGIGIGEFKDSLSINATNIKHFKNCTSI
SGDLHILPVAFRGDSFTHTPPLDPQELDILKTVKEITGFLLIQAWPENRTDLHAFENLEI
IRGRTKQHGQFSLAVVSLNITSLGLRSLKEISDGDVIISGNKNLCYANTINWKKLFGTS
GQKTKIISNRGENSCKATGQVCHALCSPEGCWGPEPRDCVSCRNVSRGRECVDKCN
LLEGEPREFVENSECIQCHPECLPQAMNITCTGRGPDNCIQCAHYIDGPHCVKTCPAG
VMGENNTLVWKYADAGHVCHLCHPNCTYGCTGPGLEGCPTNGPKIPSIATGMVGA
LLLLLVVALGIGLFMT
(CD45 scFv-CD8h-NKG2D TM-2B4-CD3ζ with signal)
SEQ ID NO: 71
MLLLVTSLLLCELPHPAFLLIPQVQLVESGGGLVQPGGSLKLSCAASGFDFSRYWMS
WVRQAPGKGLEWIGEINPTSSTINFTPSLKDKVFISRDNAKNTLYLQMSKVRSEDTAL
YYCARGNYYRYGDAMDYWGQGTSVTVSKISGGGGSGGGGSGGGGSGGGGSGGGG
SSDIVLTQSPASLAVSLGQRATISCRASKSVSTSGYSYLHWYQQKPGQPPKLLIYLAS
NLESGVPARFSGSGSGTDFTLNIHPVEEEDAATYYCQHSRELPFTFGSGTKLEIKPAPR
PPTPAPTIASQPLSLRPEACRPAAGGAVHTRGLDFACDPFFFCCFIAVAMGIRFIMVT
WRRKRKEKQSETSPKEFLTIYEDVKDLKTRRNHEQEQTFPGGGSTIYSMIQSQSSAPT
SQEPAYTLYSLIQPSRKSGSRKRNHSPSFNSTIYEVIGKSQPKAQNPARLSRKELENFD
VYSGGGRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKDTYDAL
HMQALPPR
(CD45 scFv-CD8h-NKG2D TM-2B4-CD3ζ)
SEQ ID NO: 72
QVQLVESGGGLVQPGGSLKLSCAASGFDFSRYWMSWVRQAPGKGLEWIGEINPTSS
TINFTPSLKDKVFISRDNAKNTLYLQMSKVRSEDTALYYCARGNYYRYGDAMDYW
GQGTSVTVSKISGGGGSGGGGSGGGGSGGGGSGGGGSSDIVLTQSPASLAVSLGQRA
TISCRASKSVSTSGYSYLHWYQQKPGQPPKLLIYLASNLESGVPARFSGSGSGTDFTL
NIHPVEEEDAATYYCQHSRELPFTFGSGTKLEIKPAPRPPTPAPTIASQPLSLRPEACRP
AAGGAVHTRGLDFACDPFFFCCFIAVAMGIRFIIMVTWRRKRKEKQSETSPKEFLTIY
EDVKDLKTRRNHEQEQTFPGGGSTIYSMIQSQSSAPTSQEPAYTLYSLIQPSRKSGSRK
RNHSPSFNSTIYEVIGKSQPKAQNPARLSRKELENFDVYSGGGRVKFSRSADAPAYQ
QGQNQLYNELNLGRREEYDVLDKRRGRDPEMGGKPRRKNPQEGLYNELQKDKMA
EAYSEIGMKGERRRGKGHDGLYQGLSTATKDTYDALHMQALPPR
(CD45 scFv-CD8h-NKG2D TM-2B4-CD3ζ with signal and EGFRt)
SEQ ID NO: 73
MLLLVTSLLLCELPHPAFLLIPQVQLVESGGGLVQPGGSLKLSCAASGFDFSRYWMS
WVRQAPGKGLEWIGEINPTSSTINFTPSLKDKVFISRDNAKNTLYLQMSKVRSEDTAL
YYCARGNYYRYGDAMDYWGQGTSVTVSKISGGGGSGGGGSGGGGSGGGGSGGGG
SSDIVLTQSPASLAVSLGQRATISCRASKSVSTSGYSYLHWYQQKPGQPPKLLIYLAS
NLESGVPARFSGSGSGTDFTLNIHPVEEEDAATYYCQHSRELPFTFGSGTKLEIKPAPR
PPTPAPTIASQPLSLRPEACRPAAGGAVHTRGLDFACDPFFFCCFIAVAMGIRFIIMVT
WRRKRKEKQSETSPKEFLTIYEDVKDLKTRRNHEQEQTFPGGGSTIYSMIQSQSSAPT
SQEPA YTLYSLIQPSRKSGSRKRNHSPSFNSTIYEVIGKSQPKAQNPARLSRKELENFD
VYSGGGRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKDTYDAL
HMQALPPRLEGGGEGRGSLLTCGDVEENPGPRMLLLVTSLLLCELPHPAFLLIPRKVC
NGIGIGEFKDSLSINATNIKHFKNCTSISGDLHILPVAFRGDSFTHTPPLDPQELDILKT
VKEITGFLLIQAWPENRTDLHAFENLEIIRGRTKQHGQFSLAVVSLNITSLGLRSLKEIS
DGDVIISGNKNLCYANTINWKKLFGTSGQKTKIISNRGENSCKATGQVCHALCSPEG
CWGPEPRDCVSCRNVSRGRECVDKCNLLEGEPREFVENSECIQCHPECLPQAMNITC
TGRGPDNCIQCAHYIDGPHCVKTCPAGVMGENNTLVWKYADAGHVCHLCHPNCTY
GCTGPGLEGCPTNGPKIPSIATGMVGALLLLLVVALGIGLFMT
(CD45 scFv-CD8h-CD8 TM-41BB-CD3ζ with signal)
SEQ ID NO: 74
MLLLVTSLLLCELPHPAFLLIPQVQLVESGGGLVQPGGSLKLSCAASGFDFSRYWMS
WVRQAPGKGLEWIGEINPTSSTINFTPSLKDKVFISRDNAKNTLYLQMSKVRSEDTAL
YYCARGNYYRYGDAMDYWGQGTSVTVSKISGGGGSGGGGSGGGGSGGGGSGGGG
SSDIVLTQSPASLAVSLGQRATISCRASKSVSTSGYSYLHWYQQKPGQPPKLLIYLAS
NLESGVPARFSGSGSGTDFTLNIHPVEEEDAATYYCQHSRELPFTFGSGTKLEIKPAPR
PPTPAPTIASQPLSLRPEACRPAAGGAVHTRGLDFACDIYIWAPLAGTCGVLLLSLVIT
KRGRKKLLYIFKQPFMRPVQTTQEEDGCSCRFPEEEEGGCELGGGRVKFSRSADAPA
YQQGQNQLYNELNLGRREEYDVLDKRRGRDPEMGGKPRRKNPQEGLYNELQKDK
MAEAYSEIGMKGERRRGKGHDGLYQGLSTATKDTYDALHMQALPPR
(CD45 scFv-CD8h-CD8 TM-41BB-CD3ζ)
SEQ ID NO: 75
QVQLVESGGGLVQPGGSLKLSCAASGFDFSRYWMSWVRQAPGKGLEWIGEINPTSS
TINFTPSLKDKVFISRDNAKNTLYLQMSKVRSEDTALYYCARGNYYRYGDAMDYW
GQGTSVTVSKISGGGGSGGGGSGGGGSGGGGSGGGGSSDIVLTQSPASLAVSLGQRA
TISCRASKSVSTSGYSYLHWYQQKPGQPPKLLIYLASNLESGVPARFSGSGSGTDFTL
NIHPVEEEDAATYYCQHSRELPFTFGSGTKLEIKPAPRPPTPAPTIASQPLSLRPEACRP
AAGGAVHTRGLDFACDIYIWAPLAGTCGVLLLSLVITKRGRKKLLYIFKQPFMRPVQ
TTQEEDGCSCRFPEEEEGGCELGGGRVKFSRSADAPAYQQGQNQLYNELNLGRREE
YDVLDKRRGRDPEMGGKPRRKNPQEGLYNELQKDKMAEAYSEIGMKGERRRGKG
HDGLYQGLSTATKDTYDALHMQALPPR
(CD45 scFv-CD8h-CD8 TM-41BB-CD3ζ with signal and EGFRt)
SEQ ID NO: 76
MLLLVTSLLLCELPHPAFLLIPQVQLVESGGGLVQPGGSLKLSCAASGFDFSRYWMS
WVRQAPGKGLEWIGEINPTSSTINFTPSLKDKVFISRDNAKNTLYLQMSKVRSEDTAL
YYCARGNYYRYGDAMDYWGQGTSVTVSKISGGGGSGGGGSGGGGSGGGGSGGGG
SSDIVLTQSPASLAVSLGQRATISCRASKSVSTSGYSYLHWYQQKPGQPPKLLIYLAS
NLESGVPARFSGSGSGTDFTLNIHPVEEEDAATYYCQHSRELPFTFGSGTKLEIKPAPR
PPTPAPTIASQPLSLRPEACRPAAGGAVHTRGLDFACDIYIWAPLAGTCGVLLLSLVIT
KRGRKKLLYIFKQPFMRPVQTTQEEDGCSCRFPEEEEGGCELGGGRVKFSRSADAPA
YQQGQNQLYNELNLGRREEYDVLDKRRGRDPEMGGKPRRKNPQEGLYNELQKDK
MAEAYSEIGMKGERRRGKGHDGLYQGLSTATKDTYDALHMQALPPRLEGGGEGRG
SLLTCGDVEENPGPRMLLLVTSLLLCELPHPAFLLIPRKVCNGIGIGEFKDSLSINATNI
KHFKNCTSISGDLHILPVAFRGDSFTHTPPLDPQELDILKTVKEITGFLLIQAWPENRT
DLHAFENLEIIRGRTKQHGQFSLAVVSLNITSLGLRSLKEISDGDVIISGNKNLCYANTI
NWKKLFGTSGQKTKIISNRGENSCKATGQVCHALCSPEGCWGPEPRDCVSCRNVSR
GRECVDKCNLLEGEPREFVENSECIQCHPECLPQAMNITCTGRGPDNCIQCAHYIDGP
HCVKTCPAGVMGENNTLVWKYADAGHVCHLCHPNCTYGCTGPGLEGCPTNGPKIP
SIATGMVGALLLLLVVALGIGLFMT
(CD45 scFv-IgG4(HL-CH3)-CD4 TM-41BB-CD3ζ with signal)
SEQ ID NO: 77
MLLLVTSLLLCELPHPAFLLIPQVQLVESGGGLVQPGGSLKLSCAASGFDFSRYWMS
WVRQAPGKGLEWIGEINPTSSTINFTPSLKDKVFISRDNAKNTLYLQMSKVRSEDTAL
YYCARGNYYRYGDAMDYWGQGTSVTVSKISGGGGSGGGGSGGGGSGGGGSGGGG
SSDIVLTQSPASLAVSLGQRATISCRASKSVSTSGYSYLHWYQQKPGQPPKLLIYLAS
NLESGVPARFSGSGSGTDFTLNIHPVEEEDAATYYCQHSRELPFTFGSGTKLEIKESKY
GPPCPPCPGGGSSGGGSGGQPREPQVYTLPPSQEEMTKNQVSLTCLVKGFYPSDIAVE
WESNGQPENNYKTTPPVLDSDGSFFLYSRLTVDKSRWQEGNVFSCSVMHEALHNHY
TQKSLSLSLGKMALIVLGGVAGLLLFIGLGIFFKRGRKKLLYIFKQPFMRPVQTTQEE
DGCSCRFPEEEEGGCELGGGRVKFSRSADAPAYQQGQNQLYNELNLGRREEYDVLD
KRRGRDPEMGGKPRRKNPQEGLYNELQKDKMAEAYSEIGMKGERRRGKGHDGLY
QGLSTATKDTYDALHMQALPPR
(CD45 scFv-IgG4(HL-CH3)-CD4 TM-41BB-CD3ζ)
SEQ ID NO: 78
QVQLVESGGGLVQPGGSLKLSCAASGFDFSRYWMSWVRQAPGKGLEWIGEINPTSS
TINFTPSLKDKVFISRDNAKNTLYLQMSKVRSEDTALYYCARGNYYRYGDAMDYW
GQGTSVTVSKISGGGGSGGGGSGGGGSGGGGSGGGGSSDIVLTQSPASLAVSLGQRA
TISCRASKSVSTSGYSYLHWYQQKPGQPPKLLIYLASNLESGVPARFSGSGSGTDFTL
NIHPVEEEDAATYYCQHSRELPFTFGSGTKLEIKESKYGPPCPPCPGGGSSGGGSGGQ
PREPQVYTLPPSQEEMTKNQVSLTCLVKGFYPSDIAVEWESNGQPENNYKTTPPVLD
SDGSFFLYSRLTVDKSRWQEGNVFSCSVMHEALHNHYTQKSLSLSLGKMALIVLGG
VAGLLLFIGLGIFFKRGRKKLLYIFKQPFMRPVQTTQEEDGCSCRFPEEEEGGCELGG
GRVKFSRSADAPAYQQGQNQLYNELNLGRREEYDVLDKRRGRDPEMGGKPRRKNP
QEGLYNELQKDKMAEAYSEIGMKGERRRGKGHDGLYQGLSTATKDTYDALHMQA
LPPR
(CD45 scFv-IgG4(HL-CH3)-CD4 TM-41BB-CD3ζ with signal and
EGFRt)
SEQ ID NO: 79
MLLLVTSLLLCELPHPAFLLIPQVQLVESGGGLVQPGGSLKLSCAASGFDFSRYWMS
WVRQAPGKGLEWIGEINPTSSTINFTPSLKDKVFISRDNAKNTLYLQMSKVRSEDTAL
YYCARGNYYRYGDAMDYWGQGTSVTVSKISGGGGSGGGGSGGGGSGGGGSGGGG
SSDIVLTQSPASLAVSLGQRATISCRASKSVSTSGYSYLHWYQQKPGQPPKLLIYLAS
NLESGVPARFSGSGSGTDFTLNIHPVEEEDAATYYCQHSRELPFTFGSGTKLEIKESKY
GPPCPPCPGGGSSGGGSGGQPREPQVYTLPPSQEEMTKNQVSLTCLVKGFYPSDIAVE
WESNGQPENNYKTTPPVLDSDGSFFLYSRLTVDKSRWQEGNVFSCSVMHEALHNHY
TQKSLSLSLGKMALIVLGGVAGLLLFIGLGIFFKRGRKKLLYIFKQPFMRPVQTTQEE
DGCSCRFPEEEEGGCELGGGRVKFSRSADAPAYQQGQNQLYNELNLGRREEYDVLD
KRRGRDPEMGGKPRRKNPQEGLYNELQKDKMAEAYSEIGMKGERRRGKGHDGLY
QGLSTATKDTYDALHMQALPPRLEGGGEGRGSLLTCGDVEENPGPRMLLLVTSLLL
CELPHPAFLLIPRKVCNGIGIGEFKDSLSINATNIKHFKNCTSISGDLHILPVAFRGDSFT
HTPPLDPQELDILKTVKEITGFLLIQAWPENRTDLHAFENLEIIRGRTKQHGQFSLAVV
SLNITSLGLRSLKEISDGDVIISGNKNLCYANTINWKKLFGTSGQKTKIISNRGENSCK
ATGQVCHALCSPEGCWGPEPRDCVSCRNVSRGRECVDKCNLLEGEPREFVENSECIQ
CHPECLPQAMNITCTGRGPDNCIQCAHYIDGPHCVKTCPAGVMGENNTLVWKYAD
AGHVCHLCHPNCTYGCTGPGLEGCPTNGPKIPSIATGMVGALLLLLVVALGIGLFMT
(CD45 scFv-IgG4(S228P, L235E, N297Q)-CD4 TM-41BB-CD3ζ with
signal)
SEQ ID NO: 80
MLLLVTSLLLCELPHPAFLLIPQVQLVESGGGLVQPGGSLKLSCAASGFDFSRYWMS
WVRQAPGKGLEWIGEINPTSSTINFTPSLKDKVFISRDNAKNTLYLQMSKVRSEDTAL
YYCARGNYYRYGDAMDYWGQGTSVTVSKISGGGGSGGGGSGGGGSGGGGSGGGG
SSDIVLTQSPASLAVSLGQRATISCRASKSVSTSGYSYLHWYQQKPGQPPKLLIYLAS
GPPCPPCPAPEFEGGPSVFLFPPKPKDTLMISRTPEVTCVVVDVSQEDPEVQFNWYVD
GVEVHNAKTKPREEQFQSTYRVVSVLTVLHQDWLNGKEYKCKVSNKGLPSSIEKTIS
KAKGQPREPQVYTLPPSQEEMTKNQVSLTCLVKGFYPSDIAVEWESNGQPENNYKT
TPPVLDSDGSFFLYSRLTVDKSRWQEGNVFSCSVMHEALHNHYTQKSLSLSLGKMA
LIVLGGVAGLLLFIGLGIFFKRGRKKLLYIFKQPFMRPVQTTQEEDGCSCRFPEEEEGG
CELGGGRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKDTYDAL
HMQALPPR
(CD45 scFv-IgG4(S228P, L235E, N297Q)-CD4 TM-41BB-CD3ζ)
SEQ ID NO: 81
QVQLVESGGGLVQPGGSLKLSCAASGFDFSRYWMSWVRQAPGKGLEWIGEINPTSS
TINFTPSLKDKVFISRDNAKNTLYLQMSKVRSEDTALYYCARGNYYRYGDAMDYW
GQGTSVTVSKISGGGGSGGGGSGGGGSGGGGSGGGGSSDIVLTQSPASLAVSLGQRA
TISCRASKSVSTSGYSYLHWYQQKPGQPPKLLIYLASNLESGVPARFSGSGSGTDFTL
NIHPVEEEDAATYYCQHSRELPFTFGSGTKLEIKESKYGPPCPPCPAPEFEGGPSVFLF
PPKPKDTLMISRTPEVTCVVVDVSQEDPEVQFNWYVDGVEVHNAKTKPREEQFQST
YRVVSVLTVLHQDWLNGKEYKCKVSNKGLPSSIEKTISKAKGQPREPQVYTLPPSQE
EMTKNQVSLTCLVKGFYPSDIAVEWESNGQPENNYKTTPPVLDSDGSFFLYSRLTVD
KSRWQEGNVFSCSVMHEALHNHYTQKSLSLSLGKMALIVLGGVAGLLLFIGLGIFFK
RGRKKLLYIFKQPFMRPVQTTQEEDGCSCRFPEEEEGGCELGGGRVKFSRSADAPAY
QQGQNQLYNELNLGRREEYDVLDKRRGRDPEMGGKPRRKNPQEGLYNELQKDKM
AEAYSEIGMKGERRRGKGHDGLYQGLSTATKDTYDALHMQALPPR
(CD45 scFv-gG4(S228P, L235E, N297Q)- CD4 TM-41BB-CD3ζ with
signal and EGFRt)
SEQ ID NO: 82
MLLLVTSLLLCELPHPAFLLIPQVQLVESGGGLVQPGGSLKLSCAASGFDFSRYWMS
WVRQAPGKGLEWIGEINPTSSTINFTPSLKDKVFISRDNAKNTLYLQMSKVRSEDTAL
YYCARGNYYRYGDAMDYWGQGTSVTVSKISGGGGSGGGGSGGGGSGGGGSGGGG
SSDIVLTQSPASLAVSLGQRATISCRASKSVSTSGYSYLHWYQQKPGQPPKLLIYLAS
NLESGVPARFSGSGSGTDFTLNIHPVEEEDAATYYCQHSRELPFTFGSGTKLEIKESKY
GPPCPPCPAPEFEGGPSVFLFPPKPKDTLMISRTPEVTCVVVDVSQEDPEVQFNWYVD
GVEVHNAKTKPREEQFQSTYRVVSVLTVLHQDWLNGKEYKCKVSNKGLPSSIEKTIS
KAKGQPREPQVYTLPPSQEEMTKNQVSLTCLVKGFYPSDIAVEWESNGQPENNYKT
TPPVLDSDGSFFLYSRLTVDKSRWQEGNVFSCSVMHEALHNHYTQKSLSLSLGKMA
LIVLGGVAGLLLFIGLGIFFKRGRKKLLYIFKQPFMRPVQTTQEEDGCSCRFPEEEEGG
CELGGGRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKDTYDAL
HMQALPPRLEGGGEGRGSLLTCGDVEENPGPRMLLLVTSLLLCELPHPAFLLIPRKVC
NGIGIGEFKDSLSINATNIKHFKNCTSISGDLHILPVAFRGDSFTHTPPLDPQELDILKT
VKEITGFLLIQAWPENRTDLHAFENLEIIRGRTKQHGQFSLAVVSLNITSLGLRSLKEIS
DGDVIISGNKNLCYANTINWKKLFGTSGQKTKIISNRGENSCKATGQVCHALCSPEG
CWGPEPRDCVSCRNVSRGRECVDKCNLLEGEPREFVENSECIQCHPECLPQAMNITC
TGRGPDNCIQCAHYIDGPHCVKTCPAGVMGENNTLVWKYADAGHVCHLCHPNCTY
GCTGPGLEGCPTNGPKIPSIATGMVGALLLLLVVALGIGLFMT

Cell Isolation, CD45 gene editing, CD45-CAR Lentiviral Transduction, and Ex Vivo Expansion_Leukapheresis products were obtained from consented research participants (healthy donors) under protocols approved by the City of Hope Internal Review Board (IRB). On the day of leukapheresis, peripheral blood mononuclear cells (PBMC) were isolated by density gradient centrifugation over Ficoll-Paque (GE Healthcare) followed by multiple washes in PBS/EDTA (Miltenyi Biotec). Cells were rested overnight at room temperature (RT) on a rotator, and subsequently washed and resuspended in X-VIVO T cell medium (Lonza) containing 10% FBS (complete X-VIVO). Up to 5.0×10⁹ PBMC were incubated with anti-CD14 and anti-CD25 microbeads (Miltenyi Biotec) for 30 min at RT and magnetically depleted using the CliniMACS® system (Miltenyi Biotec) according to the manufacturer's protocol and these were termed depleted PBMCs (dPBMC). dPBMC were frozen in CryoStor® CS5 (StemCell Technologies) until further processing. Tn/mem cells were prepared from dPBMC by staining with anti-CD62L microbeads(Miltenyi Biotec) and enriching CD62L+ cells using AutoMACS system..dPBMC or Tn/mem were stimulated with CD3/CD28 Dyna-beads (Thermal Fisher Scientific, Ratio of Cell to Beads is 1 to 2) in X-vivo15 medium with 10 U/mL IL2 and 0.5 ng/mL IL5. After one day, the cells were harvested and PTPRC (CD45) gene was knocked out by CRISPR-Cas9 ribonucleoprotein (RNP) system. For small scale experiment, the RNP was prepared by mixing 60 pmol Truecut Cas9 V2 protein (Thermo Fisher) and 180 pmol gRNA targeting PTPRC in 50 uL electroporation P3 buffer (Lonza) and incubate for 15 min at room temperature. The RNP solution was then mixed with 50 uL T cell suspension of 2 million cells and delivered by electroporation using 4D Nucleofector system (Lonza). After electroporation, T cells were incubated with 0.5 mL culture medium for 15 min then transferred to wells with 2 mL medium and fresh CD3/CD28 beads (Ratio of Cells to Beads is 1 to 1.)

Lentiviral transduction was performed at 2-5 days after gene editing. Briefly gene modified T cells were cultured with CD3/CD28 Dynabeads® (Life Technologies), protamine sulfate (APP Pharmaceuticals), cytokine mixture (as stated above) and desired lentivirus at a multiplicity of infection (MOI) of 1-3. Cells were then cultured in and replenished with fresh complete X-VIVO containing cytokines every 2-3 days. After 7 days, beads were magnetically removed, and cells were further expanded in complete X-VIVO containing cytokines to achieve desired cell yield. Following further expansion, cells were frozen in CryoStor® CS5 prior to in vitro functional assays and in vivo tumor models. Purity and phenotype of CAR T cells were verified by flow cytometry. We designed multiple gRNAs targeting different exons of CD45 (PTPRC) gene to knock out it. Examples used are as follows: hCD45gRNA #1_E3, AUAUUAAUUCUUACCAGUGG (SEQ ID NO: 37); hCD45gRNA #2_E8, ACUCCAUCUAAGCCAACAUG (SEQ ID NO: 38); hCD45gRNA #3_E12, CUUCUACAAAAAAUAAUCUG (SEQ ID NO: 39); hCD45gRNA #4_E25, GUGCUGGUGUUGGGCGCAC (SEQ ID NO: 45).

Flow Cytometry

T cells were harvested and stained as described previously (Jonnalagadda, M., et al., Chimeric antigen receptors with mutated IgG4 Fc spacer avoid fc receptor binding and improve T cell persistence and antitumor efficacy. Mol Ther, 2015. 23(4): p. 757-68.). T cell phenotype was examined using fluorochrome-conjugated antibodies against CD3, CD4, CD8α, CD45 (clone HI30, BC-8 or 94.1). Transgenic CAR expression was determined by staining of the truncated EGFR tag. Data were acquired on MacsQuant Analyzer 10 (Miltenyi Biotec) flow cytometers and analyzed with FlowJo (v10.6.1).

In Vitro T Cell Assays

For tumor killing assays, CAR T cells and tumor targets were co-cultured at indicated effector:tumor (E:T) ratios. To test cytotoxicity effect of CD45CAR T cells, GFP expressing tumor cells were plated in 96-well U-bottom plates at the indicated density. Effector cells (CD45KO CD45CAR T or Mock T cells) were washed, resuspended in fresh medium without cytokines and co-cultured with the indicated tumor cells for 4 hours (short term) or 48 hours (long term). Cytotoxicity was routinely evaluated by flow cytometry with enumeration of GFP+DAPI-tumor cells for viable GFP-expressing tumor cells. For primary PBMC, viable T cells (CD3+), B cells (CD19+) and myeloid cells (CD11b+) were analyzed by staining with lineage specific markers.

To test for degranulation activity, CAR T or control T cells were incubated with tumor cells for five hours in the presence of CD107a antibody and GolgiStop protein transport inhibitor (BD Biosciences). After the co-culture, cells were harvested, fixed, permeabilized, and stained for intracellular cytokines. Degranulation (CD107a staining) and intracellular cytokine staining (e.g. IFNγ) were examined by flow cytometry.

In Vivo Tumor Studies

All animal experiments were performed under protocols approved by the City of Hope Institutional Animal Care and Use Committee. Tumor xenograft models were generated using 6 to 8 week-old NOD/SCID/IL2R−/− (NSG) mice as previously described (Jackson Laboratory) [Urak, R., et al., Ex vivo Akt inhibition promotes the generation of potent CD19CAR T cells foradoptive immunotherapy. J Immunother Cancer, 2017. 5:26]. Briefly, on day 0, ffLuc+MV4-11 cells (1×10⁶) were injected intravenously (i.v.) into the NSG mice. After 5 days, mice were then treated with CAR T cells or mock T cells as described for each experiment. Tumor growth was determined by in vivo bio-photonic imaging using a Xenogen IVIS 100. Mice were also monitored for survival, with euthanasia applied according to the American Veterinary Medical Association Guidelines.

### Example 1: Generation of CD45KO CD45 CAR T cells

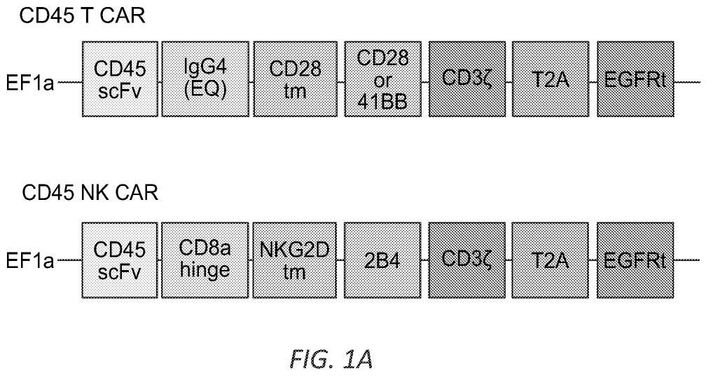

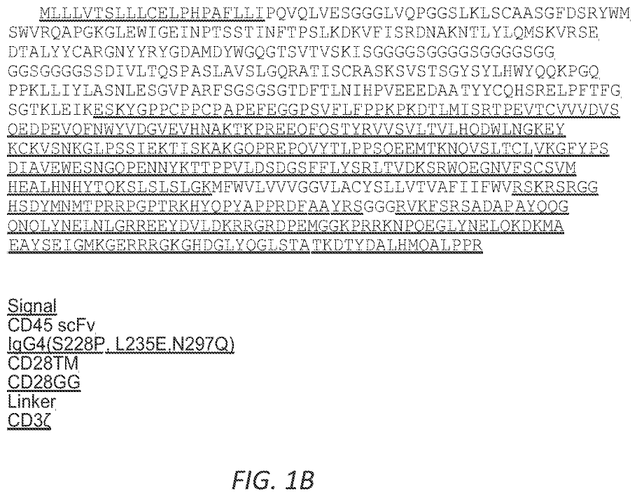

We utilized the single chain variable fragment (scFv) sequence of an anti-CD45 antibody clone BC-8, which already shown good profiles of safety and specificity [Mawad, R., et al., Radiolabeled anti-CD45 antibody with reduced-intensity conditioning and allogeneic transplantation for younger patients with advanced acute myeloid leukemia or myelodysplastic syndrome. Biol Blood Marrow Transplant, 2014. 20(9):1363-8; Lin, Y., et al., A genetically engineered anti-CD45 single-chain antibody-streptavidin fusion protein for pretargeted radioimmunotherapy of hematologic malignancies. Cancer Res, 2006. 66(7):3884-92; Orozco, J. J., J. Zeller, and J. M. Pagel, Radiolabeled antibodies directed at CD45 for conditioning prior to allogeneic transplantation in acute myeloid leukemia and myelodysplastic syndrome. Ther Adv Hematol, 2012. 3(1):5-16]. The CD45 CAR construct for T cells is composed with anti-CD45 scfv domain, an IgG4 spacer with two point-mutations (L235E and N297Q) within the CH2 region, a CD28GG costimulatory domain, CD3ζ, and a truncated human epidermal growth factor receptor (huEGFRt) as a marker (FIG. 1A-1B). The CAR construct can also use other costimulatory domains such as 41BB, other spacer domains and transmembrane domains such as CD8 hinge and CD4 transmembrane domain. For CAR constructs for NK cells, the transmembrane domain can be the NKG2D transmembrane domain and the costimulatory domain can be the 2B4 costimulatory domain.

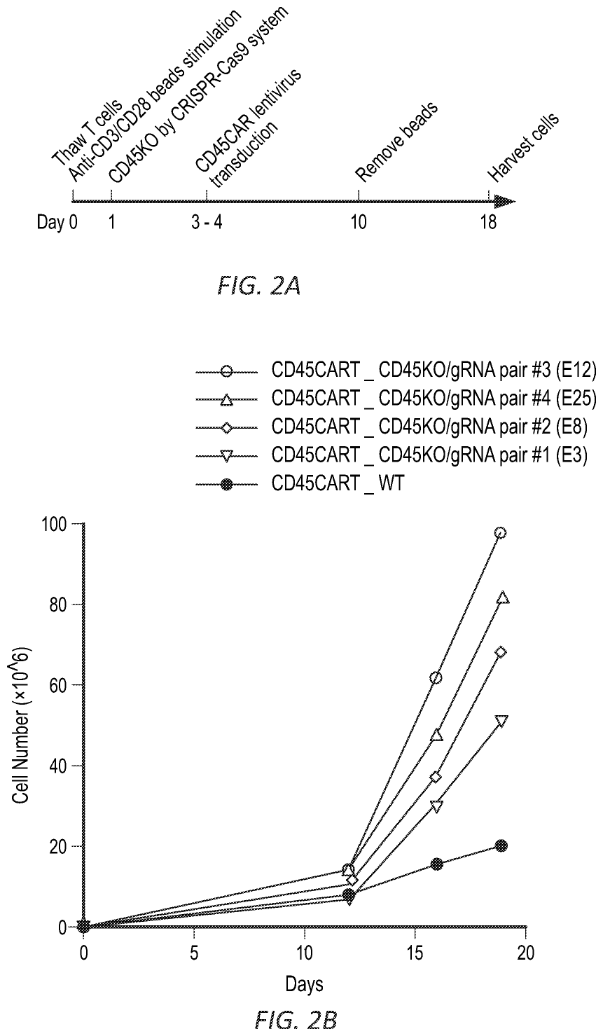

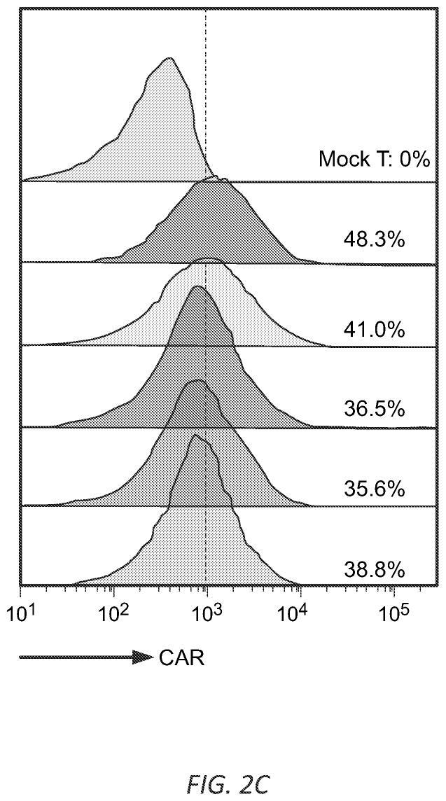

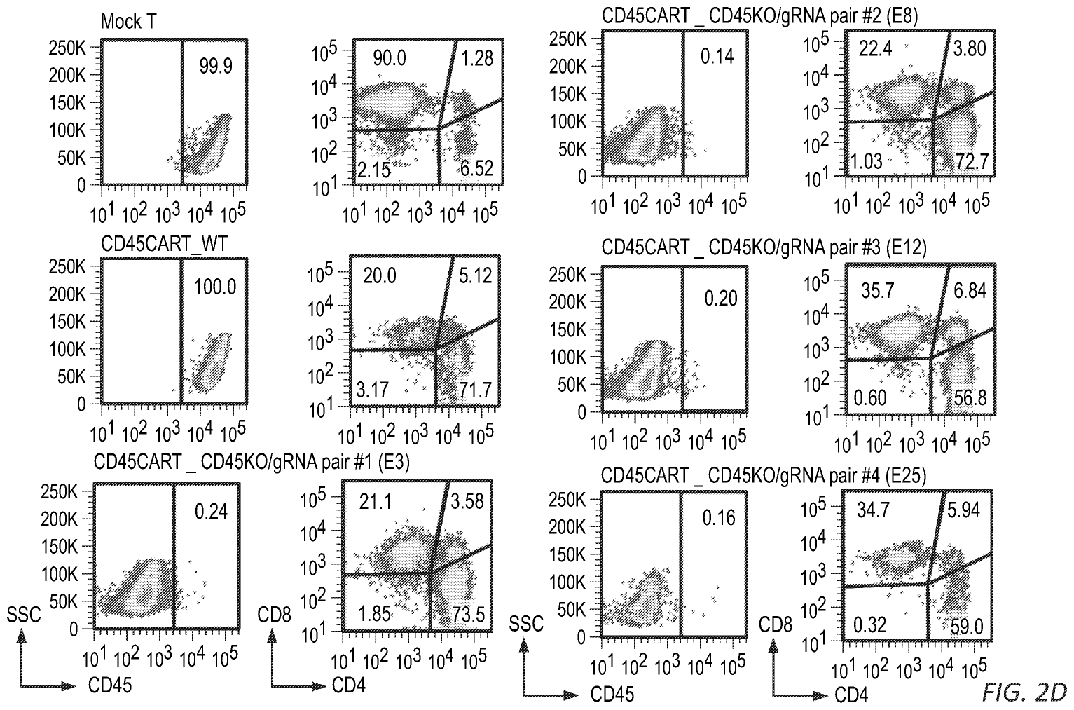

CD45KO CD45CAR T cells can be prepare from, for example, PBMC, dPBMC (PBMC with depletion of CD14+ and CD25+ cells), Tn/mem (naïve and memory T cells, CD62L+ enriched from dPBMC), or Tcm (central memory T cells). In this case, CD45KO CD45CAR T cells were generated from Tn/mem cells (FIG. 2A). A number of different gRNAs targeting different exons of PTPRC gene were used knock out PTPRC gene. The generated CD45CAR T cells with PTPRC gene knock out demonstrated better proliferation capacity and generated more cells compared to WT CD45CAR T cells (FIG. 2B), in which CD45CAR can cause a fratricidal effect. There was a CAR positive percentage range from 35.5% to 48.3% with MOI of 2 for lenti virus transduction (FIG. 2C). Surface marker analysis by flow cytometry showed that CD45KO CD45CAR T cells had no detectable cell surface CD45 expression (FIG. 2D). Moreover, the relative CD4+ and CD8+ population was not significantly altered (FIG. 2D).

### Example 2: Validation that CD45 CAR T Cells Selectively Target CD45-Positive Cells In Vitro

To determine whether CD45 CAR T cells demonstrate selective activity against CD45-positive cancer and noncancerous cells, the CD45 CAR T cells were grown in presence of either CD45-positive cells.

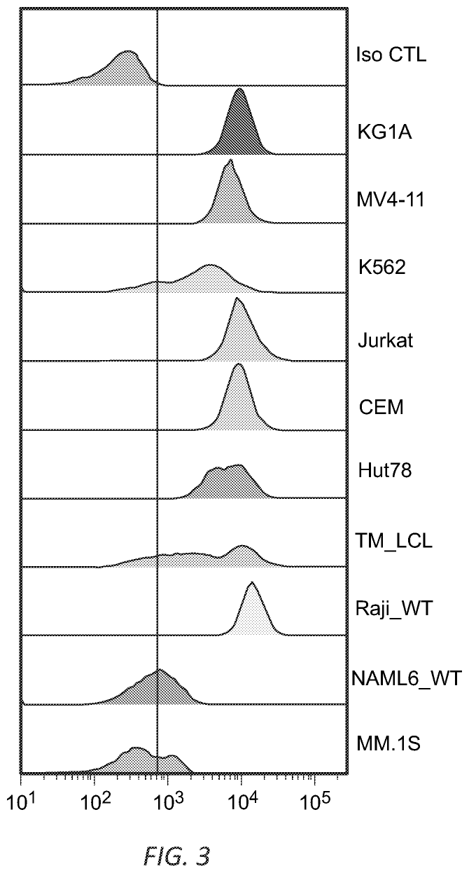

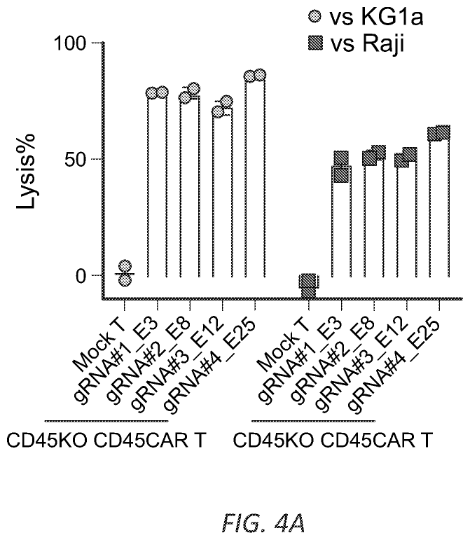

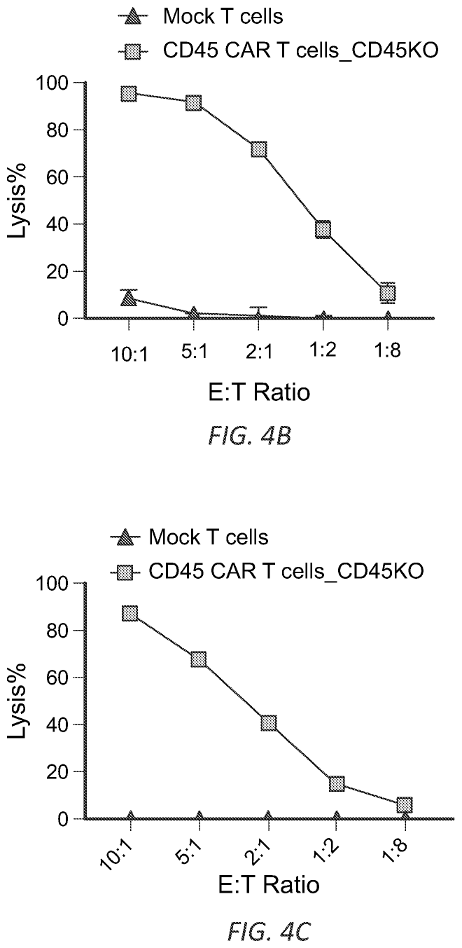

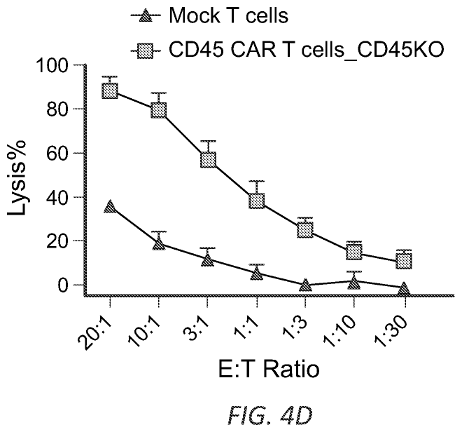

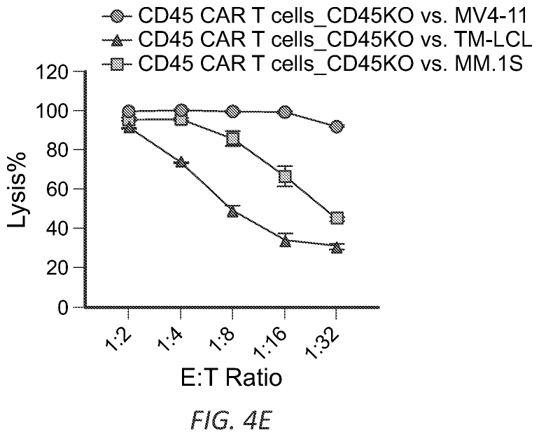

As shown in FIG. 3, CD45 is widely expressed on different hematopoietic malignant cells, including acute myeloid leukemia (KG1A, MV4-11, K562), T cell leukemia and lymphoma (Jurkat, CEM, Hut78), B cell leukemia and lymphoma (TM-LCL, Raji, NALM6) and Multiple Myeloma (MM.1S). It was also well established that CD45 is a hematopoietic specific marker in healthy physiological condition [Rheinlander, A., B. Schraven, and U. Bommhardt, CD45 in human physiology and clinical medicine. Immunol Lett, 2018. 196:22-32]. By coculturing with CD45+KG1A and Raji cells, all CD45KO CD45CAR T cells demonstrated potent antigen specific cytotoxicity while mock T cells did not (FIG. 4A).

The gRNA #3 (target PTPRC exon 12) CD45 knock out cells were used for further functional characterization. The CD45KO CD45CAR T cells demonstrated potent cytotoxicity against AML (KG1a, MV4-11) (FIGS. 4B and 4E), B-ALL (Raji, TM-LCL) (FIG. 4C), blast crisis CML (KCL22M with T315I mutation) (FIG. 4D) and multiple myeloma (MM.1 S) (FIG. 4E).

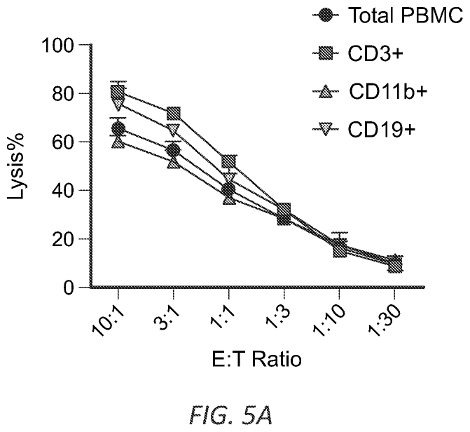

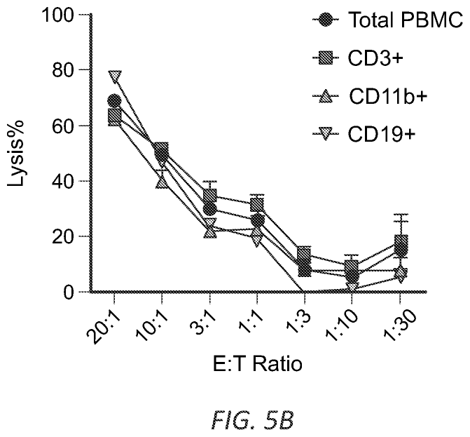

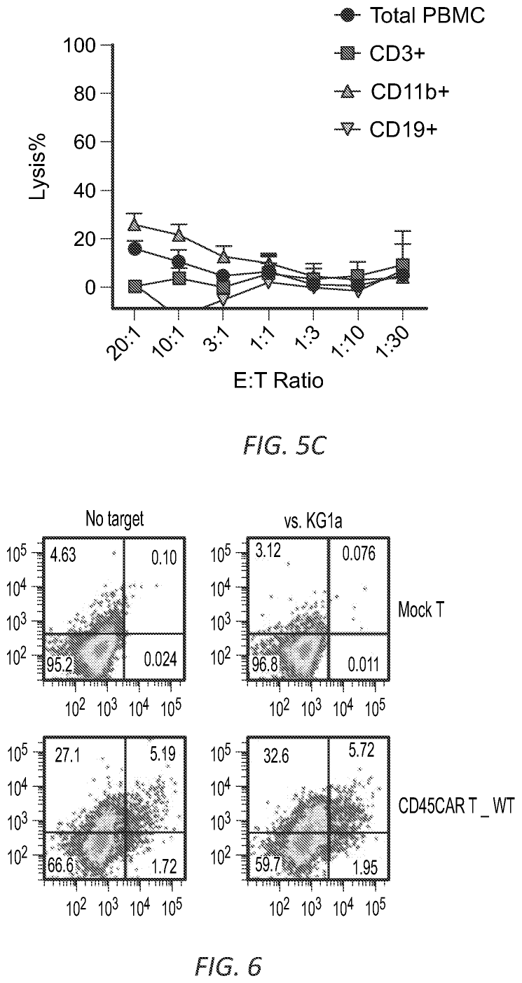

By co-culturing with PBMC from healthy donor, CD45KO CD45CAR T cells were shown to eliminate healthy myeloid cells (CD11b+), B cells (CD19+) and T cells (CD3+) (FIGS. 5A and 5B), while mock T cells did not (FIG. 5C). This functional phenotype indicated potential application of CD45KO CD45 CAR T cells for HCT conditioning.

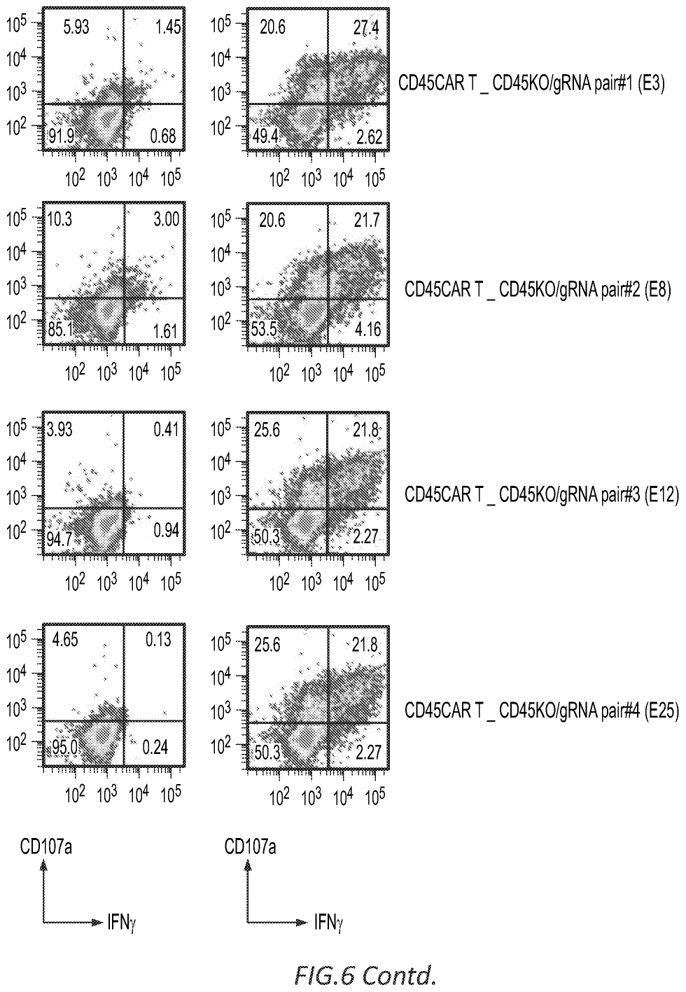

The CD45KO CD45CAR T cells also demonstrated potent antigen specific degranulation activity and IFNγ secretion (FIG. 6).

### Example 3: Validation that CD45 CAR T Cells Delivered In Vivo in a Mouse Model Exhibit Potent Anti-Tumor Activity and Confer Extended Lifespan to the Mice

To evaluate in vivo efficacy of CD45 CAR T cells to selectively target CD45-positive cells in the AML model, CD45 CAR T cells were delivered and tumor size and survival was evaluated over time.

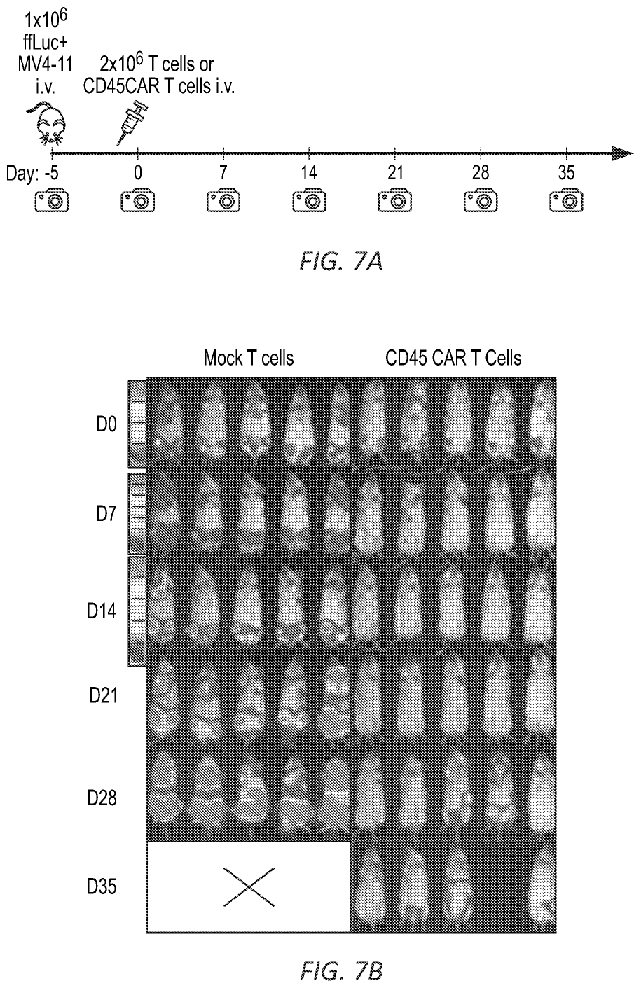

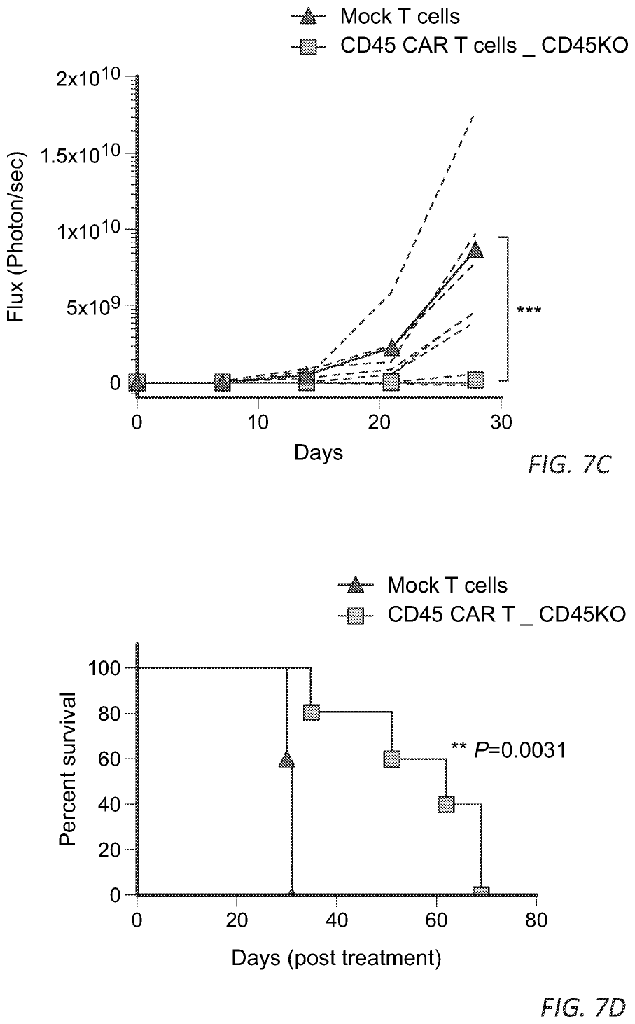

To further evaluate the in vivo activity, we tested in a tumor xenograft mouse model with MV4-11 AML cells (FIG. 7A). CD45KO CD45 CAR T cells treatment significantly eliminated tumor engrafts and prolonged the survival of the mice (FIGS. 7B-7D).

### Example 4: Validation that CD45KO CAR T Cells Targeted to Other Antigens Benefit from Enhanced Features

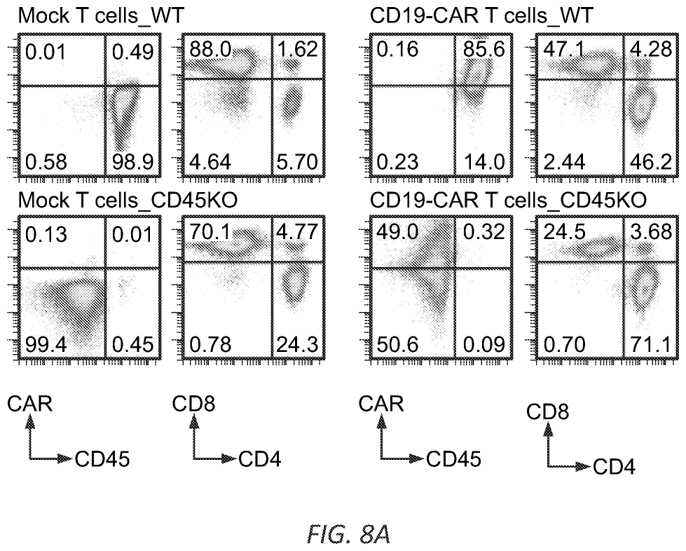

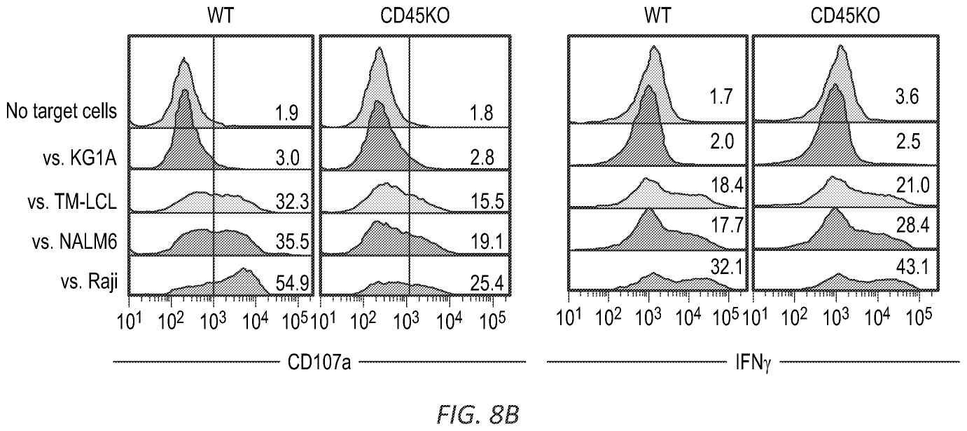

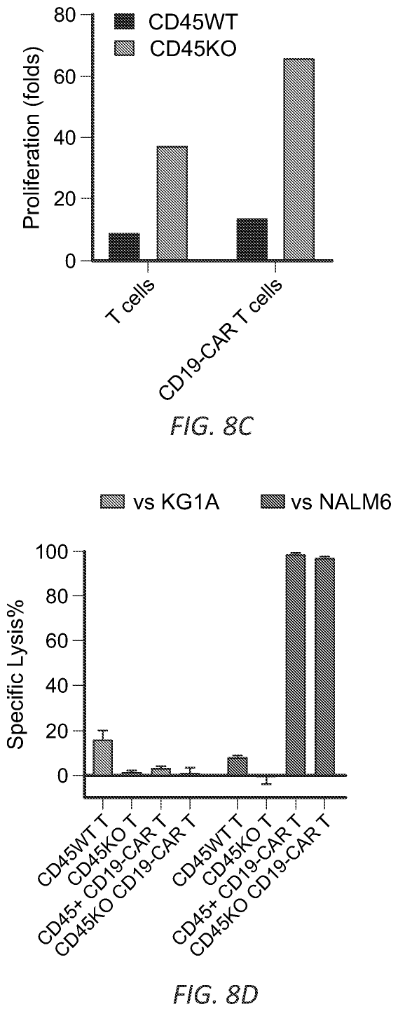

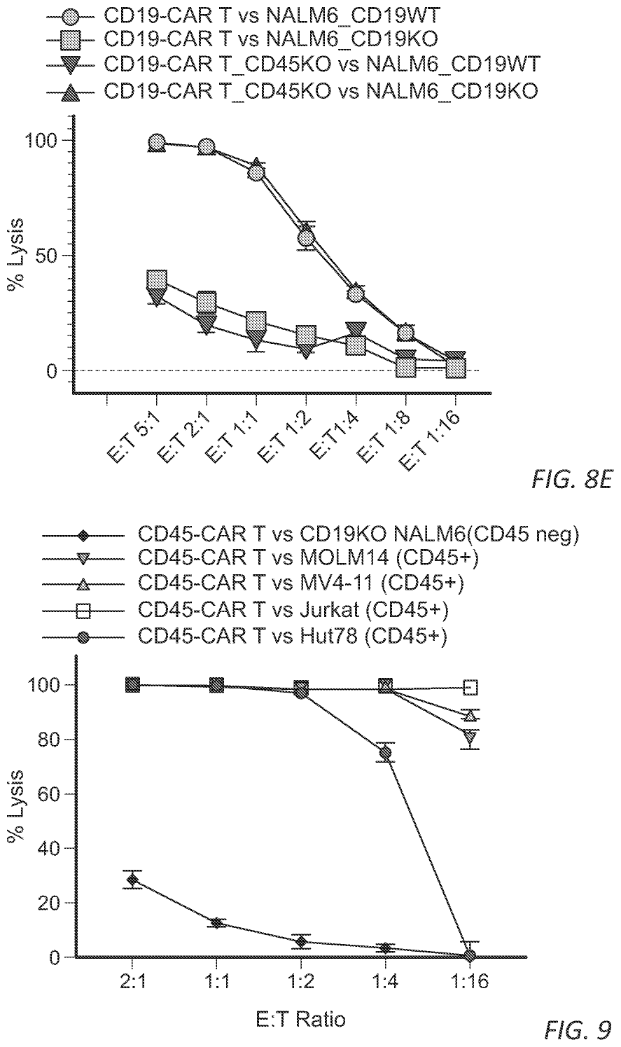

CD45 is reported to play key roles in T cell development and function regulation in both negative and positive way [Alexander, D. R., The CD45 tyrosine phosphatase: a positive and negative regulator of immune cell function. Semin Immunol, 2000. 12(4):349-59; Cho, J. H., et al., CD45-mediated control of TCR tuning in naive and memory CD8(+) T cells. Nat Commun, 2016. 7:13373; Virts, E. L., O. Diago, and W. C. Raschke, A CD45 minigene restores regulated isoform expression and immune function in CD45-deficient mice: therapeutic implications for human CD45-null severe combined immunodeficiency. Blood, 2003. 101(3):849-55]. However, the role of CD45 in CAR T cells is not well studied. We explored the functions of CD45 on CD19-CAR T cells by knocking out CD45. As shown in FIG. 8A, knocking out CD45 on CD19-CAR T cells or mock T cells increased CD4+ population with expense of CD8+ population. CD45KO CD19-CAR T cells demonstrated potent degranulation, IFNγ secretion activity (FIG. 8B) and comparable antigen specific cytotoxicity (FIG. 8D). As shown in FIG. 8E, which depicts the results of a 4 hr cytotoxicity assay using wt or CD45KO CD19-CAR T cells against WT NALM6 or CD19KO NALM6, knockout of CD45 did not impair antigen specific cytotoxicity of CD19-CAR T cells. The CD45 knockout enhanced the proliferation of T cells and CD19CAR T cells (FIG. 8C). This phenotype suggests various CAR T cells, e.g., CAR T cells targeted to CD19 may benefit from knockout or knockdown of CD45.

### Example 5: Myeloid/Lymphoid Ablation Effect of CD45 CAR T Cells in Humanized Mouse Model with Human PBMC or HSC Engraftments

In some circumstances it is desirable to reduce or eliminate myeloid and/or lymphoid cells in vivo. Experiments will be conducted in a humanized mouse model to measure depletion of myeloid and lymphoid cells as a function of treatment with CD45KO CD45 CAR T cells and CD45KO CD45 CAR NK cells. Results will show a reduction of myeloid and/or lymphoid cells and in increase of success of PBMC and/or HCS (hematopoietic stem cells) engraftment with CD45 CAR T cells and/or CD45KO CD45 CAR NK cells treatment.

### Example 6: CD45KO CD45CAR T Cells have Antigen-Specific Anti-Tumor and Myeloid-Ablation and Lymphoid-Ablation Activity

Killing assays demonstrated that CD45KO CD45CAR T cells have antigen specific anti-tumor and myeloid-ablation and lymphoid-ablation activity. Luciferase-based Cytotoxicity Assay (LCA) of CD45KO CD45-CAR T cells against different CD45+ target cells (MOLM14, MV4-11, Jurkat, and Hut78) with 48-hour co-culture in different Effector (E): Target (T) ratios showed effective killing of the CD45+ target cells (FIG. 9).

### Example 7: Validation of iCasp9-CD45-CAR Expression and Function

To test the effects of a suicide switch as a safety precaution, experiments were conducted to investigate the induced cell depletion effect of rimiducid on iCasp9-CD45-CAR lentivirus transduced cells. iCasp9 can be activated by a specific chemical inducer of dimerization (CID) such as rimiducid, leading to efficient elimination of iCasp9 engineered cell.

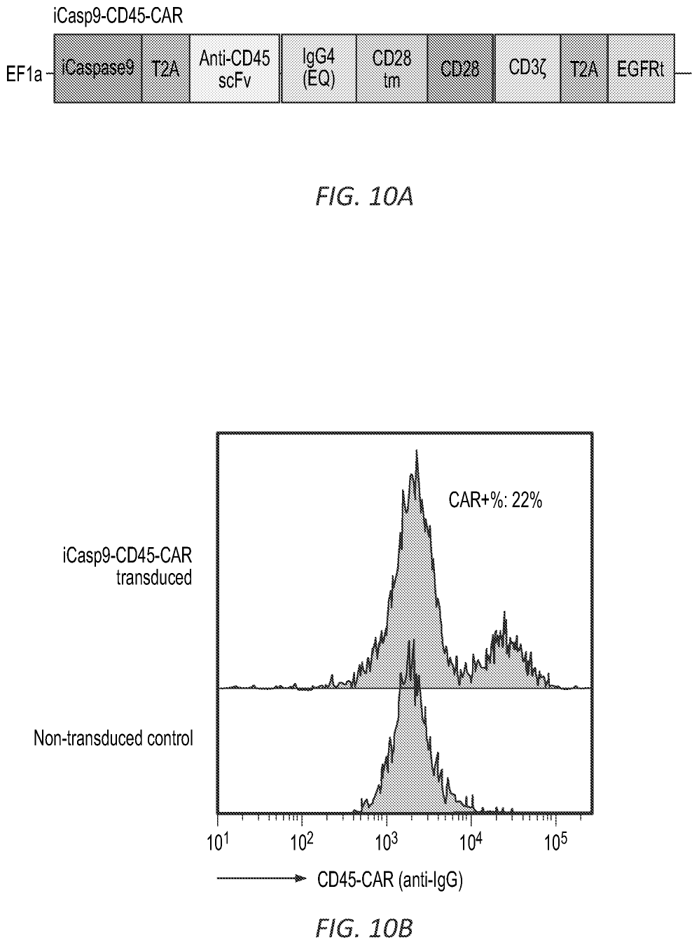

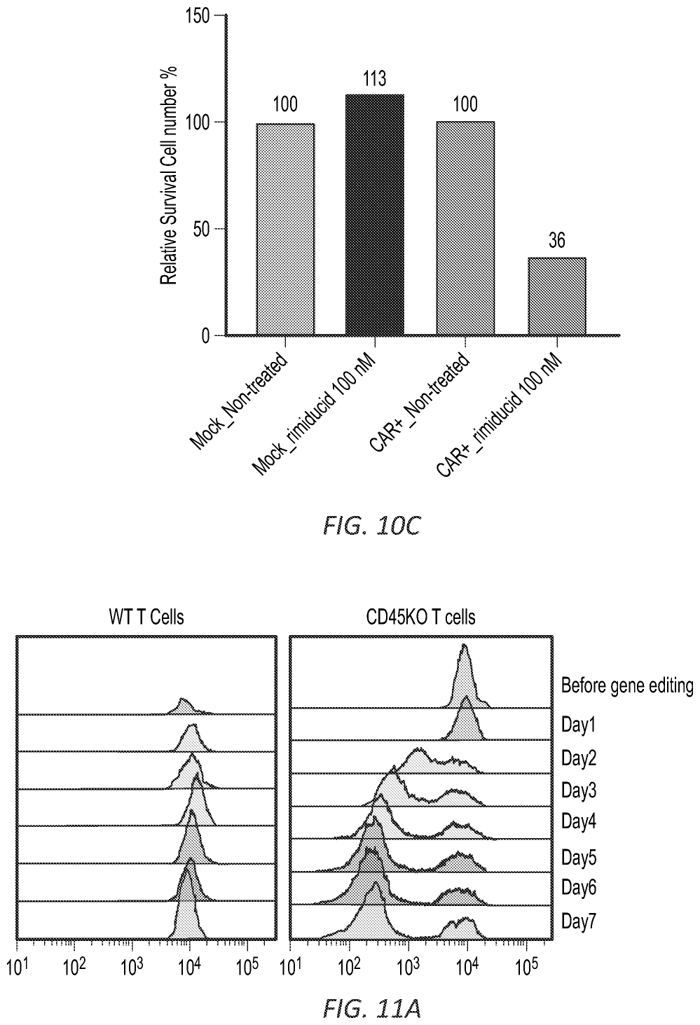

The design of an iCasp9-CD45-CAR construct containing iCaspase9 is shown in FIG. 10A. The order of iCaspase 9 and CD45 scFv components can be switched (e.g., promoter-CD45CAR-T2A-iCaspase9-etc). HT1080 cells were transduced by lentivirus. Flow cytometry showed 22% of the transduced T cells expressed CD45 CAR (FIG. 10B). Transduced HT1080 cells were seeded in 24 well plate and treated with 100 μM ramiducid for 24h normalized and compared to non-treated group, and the relative survival cell number was quantified (FIG. 10C). Treating the iCasp9-CD45-CAR cells with ramiducid reduced cell survival to 36% (FIG. 10C).

### Example 8: Validation of CD45 Depletion on T Cells Following CRISPR/Cas9 Gene Editing

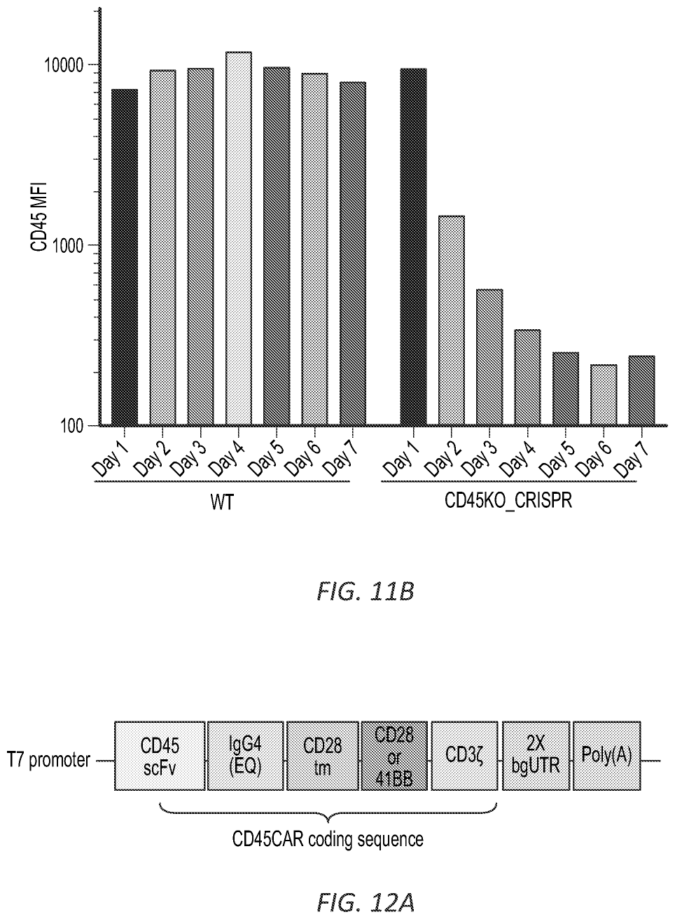

Experiments were then conducted to quantify the CD45 protein expression on wild type and CD45 knock out T cells after PTPRC (CD45) gene knockout by CRISPR/Cas9 gene editing. hCD45gRNA #3_E12, CUUCUACAAAAAAUAAUCUG (SEQ ID NO: 39) was used for this experiment. This experiment demonstrated the decreasing expression of CD45 via flow cytometry (FIG. 11A) and quantified MFI (FIG. 11B) after CRISPR/cas9 mediated CD45KO, which provided evidence that transduction CD45-CAR after 5 days following CRISPR can avoid fratricide effect.

### Example 9: CD45KO CD45CAR T Cells Generated by mRNA Transduction/iCaspase9 for Generating mRNA by In Vitro Transcription Method/mRNA Electroporated T Cells can Preserve Transitory Expression for 2 Weeks

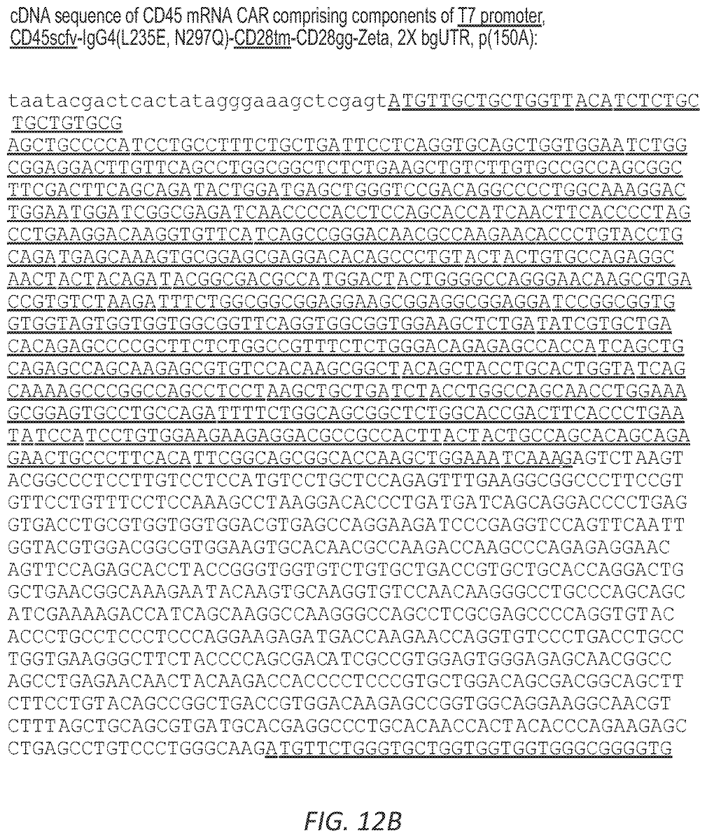

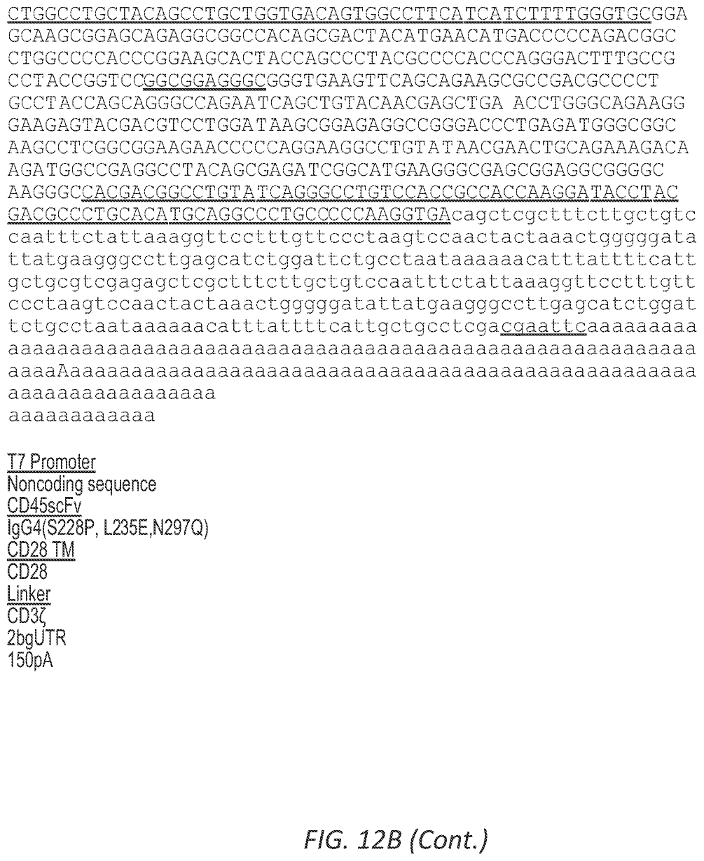

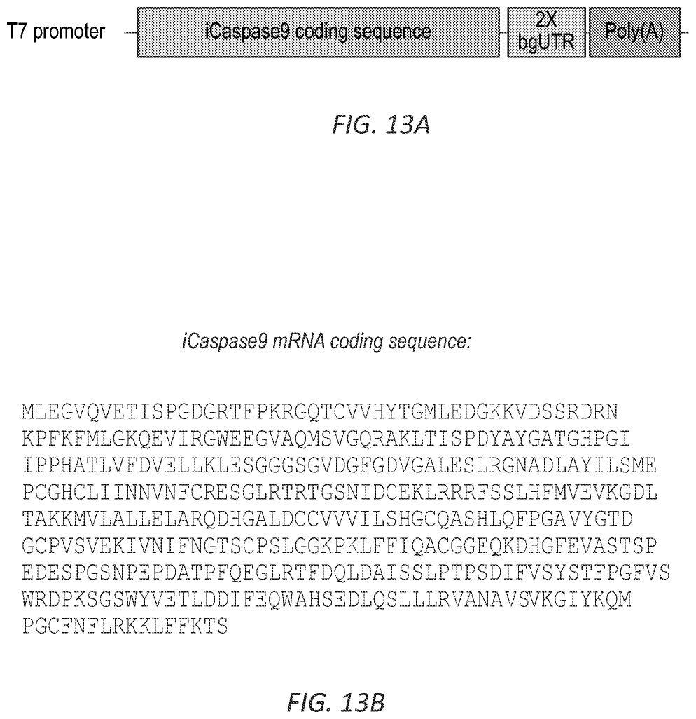

The design of a CD45 mRNA CAR is shown in FIG. 12A. The sequence of the CD45 mRNA CAR is shown in FIG. 12B. CD45KO CD45CAR T cells are used here but it works the same for a CD45KO CD45CAR NK cells (data not shown). The design of an iCaspase 9 mRNA is shown in FIG. 13A. The sequence of the iCaspase 9 mRNA is shown in FIG. 13B. The iCaspase 9 mRNA is used as a safety switch construct.

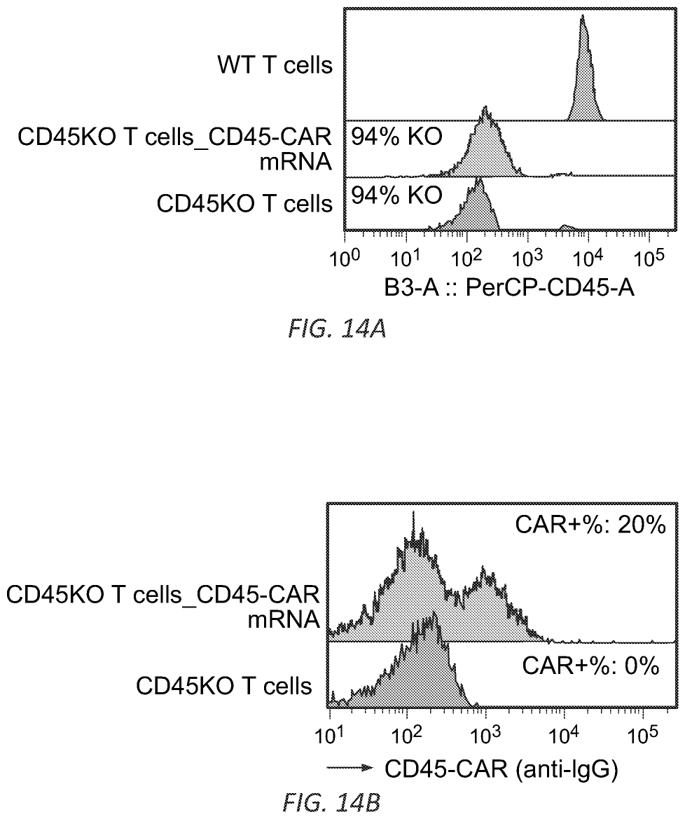

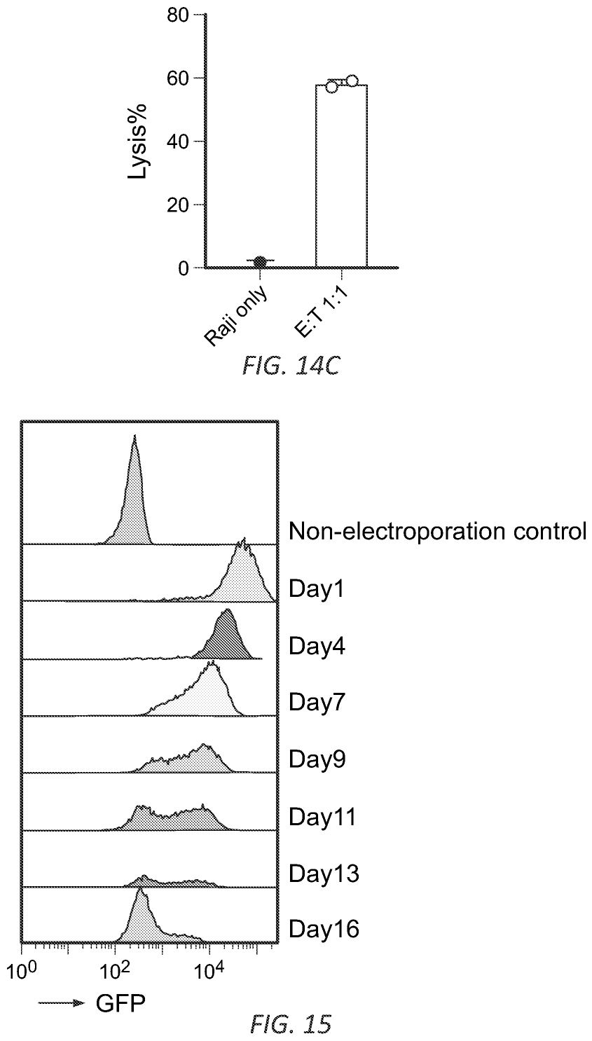

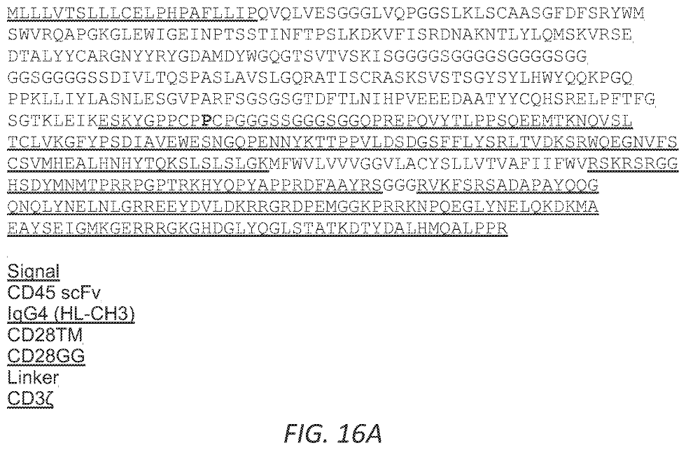

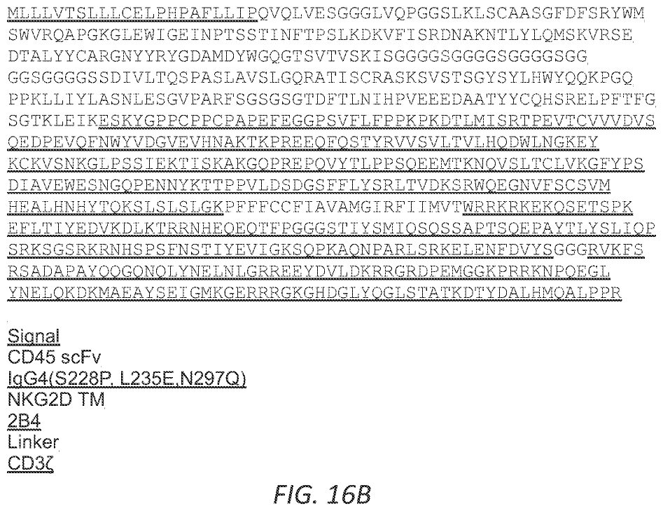

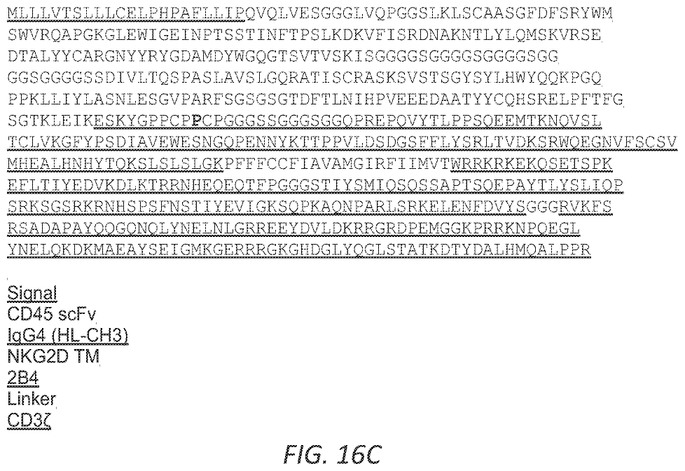

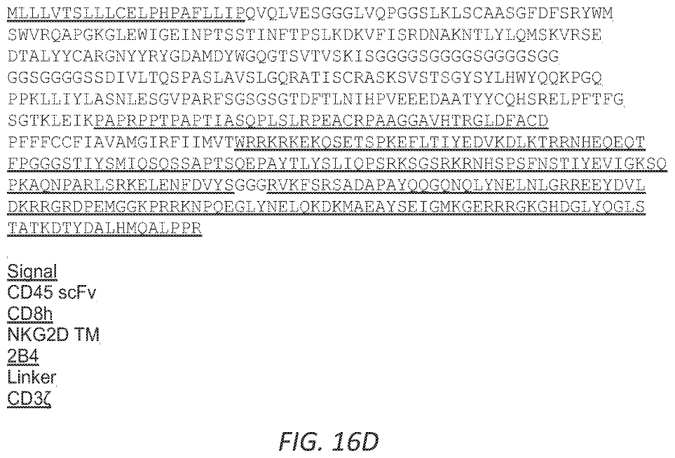

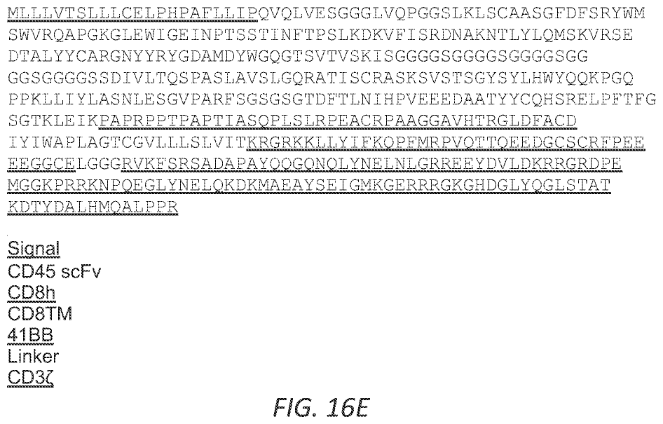

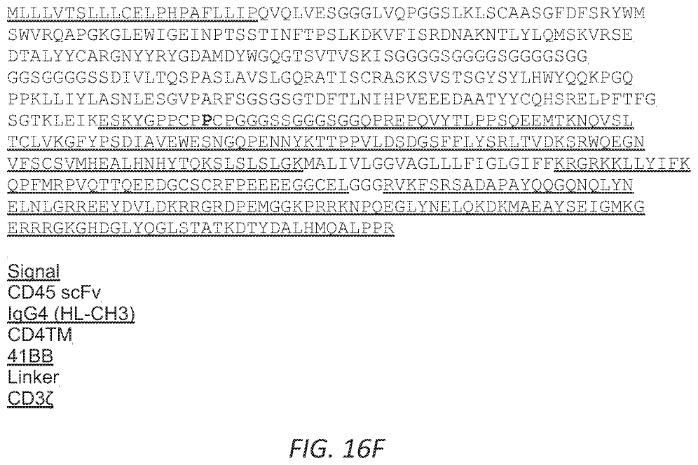

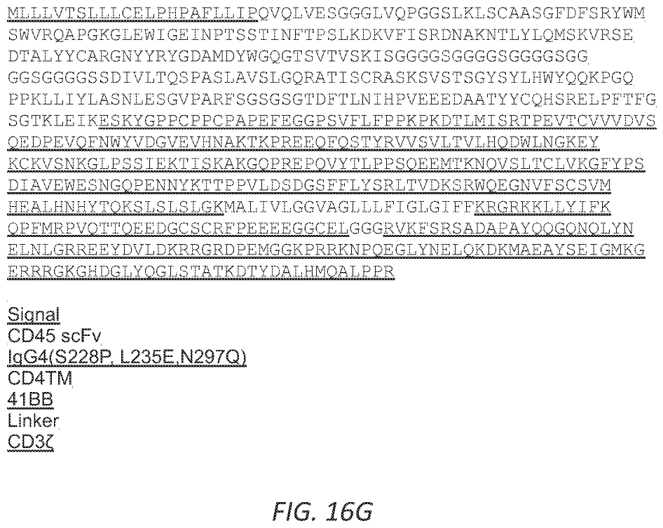

Experiments were then conducted to investigate the characteristics of CD45KO CD45CAR T cells generated by mRNA transduction. Flow cytometry probed the CD45 expression profile on wild type T cells, CD45KO T cells, and CD45KO CD45CAR T cells and showed CD45 was effectively knocked out (FIG. 14A). The CAR expression profile of CD45KO CD45CAR T cells 24h after mRNA electroporation shows 20% of the cells expressed the CAR (FIG. 14B).

T cells cultured for 7 days were transduced via electroporation with GFP mRNA in a dose of 2.5 ug/million and the expression level of GFP expression was tracked by flow cytometry. This data demonstrated that GFP mRNA can express GFP protein for about 2 weeks. Importantly, mRNA electroporated T cells preserved expression for 2 weeks indicates the feasibility of mRNA CD45CAR T cells with transitional expression as a strategy to make these CAR T and NK cells safer for patients.

### Other Embodiments

It is to be understood that while the invention has been described in conjunction with the detailed description thereof, the foregoing description is intended to illustrate and not limit the scope of the invention.

What is claimed is:

1. A nucleic acid molecule comprising a nucleotide sequence encoding a chimeric antigen receptor (CAR) or a polypeptide, wherein the chimeric antigen receptor or polypeptide comprises: an scFv targeting CD45, a spacer, a transmembrane domain, a co-stimulatory domain, and a CD3 ζ signaling domain.

2. The nucleic acid molecule of claim 1, wherein the scFv comprises the amino acid sequence of SEQ ID NO:1 or variant thereof having 1-5 amino acid modifications.

3. The nucleic acid molecule of claim 1, wherein the scFv comprises the amino acid sequence of SEQ ID NO:32 or variant thereof having 1-5 amino acid modifications and the amino acid sequence of SEQ ID NO: 33 or variant thereof having 1-5 amino acid modifications.

4. The nucleic acid molecule of claim 1, wherein the transmembrane domain is selected from the group consisting of: a CD4 transmembrane domain or variant thereof having 1-5 amino acid modifications, a CD8 transmembrane domain or variant thereof having 1-5 amino acid modifications, a CD28 transmembrane domain or a variant thereof having 1-5 amino acid modifications, and a NKG2D transmembrane domain or a variant thereof having 1-5 amino acid modifications.

5. The nucleic acid molecule of claim 1, wherein the transmembrane domain is a CD4 transmembrane domain, a CD8 transmembrane domain, a CD28 transmembrane domain or a NKG2D transmembrane domain.

6. The nucleic acid molecule of claim 1, wherein the costimulatory domain is a CD28, 4-1BB, or a 2B4 costimulatory domain.

7. The nucleic acid molecule of claim 1, wherein the costimulatory domain comprises the amino acid sequence of any of SEQ ID NOs:22-25 and 54 or a variant thereof having 1-5 amino acid modifications.

8. The nucleic acid molecule of claim 1, wherein the CD3ζ signaling domain comprises the amino acid sequence of SEQ ID NO:21.

9. The nucleic acid molecule of claim 1, wherein a linker of 3 to 15 amino acids is located between the costimulatory domain and the CD3 ζ signaling domain or variant thereof.

10. The nucleic acid molecule of claim 1, wherein the spacer comprises any one of SEQ ID NOs:2-12 or a variant thereof having 1-5 amino acid modifications.

11. The nucleic acid molecule of claim 1, wherein the CAR or the polypeptide comprises the amino acid sequence of SEQ ID NO:29 or 30, or a variant thereof having 1-5 amino acid modifications.

12. The nucleic acid molecule of claim 1, which is an mRNA molecule.

13. An expression vector comprising the nucleic acid molecule of claim 1.

14. A population of human T cells harboring the mRNA molecule of claim 12.

15. A population of human T cells transduced by a vector comprising the nucleic acid molecule of claim 1.

16. The population of human T cells of claim 15, wherein the population of human T cells comprise central memory T cells, naive memory T cells, CD4+ cells and CD8+ cells enriched from PBMC cells, T cells isolated via negative depletion, or PBMC substantially depleted for CD25+ cells and CD14+ cells.

17. The population of T cells of claim 15, wherein CD45 (PTPRC) is knocked out, knocked down, or mutated.

17a. The population of T cells of claim 17, wherein the mutation is a deletion that eliminates CD45 expression.

18. The population of T cells of claim 17, wherein CD45 (PTPRC) is knocked out, knocked down, or mutated by CRISPR-Cas9 or TALEN system.

19. A population of human NK cells transduced by a vector comprising the nucleic acid molecule of claim 1 or harboring the mRNA molecule of claim 12.

20. The population of NK cells of claim 19, wherein CD45 (PTPRC) is knocked out, knocked down, or mutated.

20a. The population of NK cells of claim 20, wherein the mutation is a deletion that eliminates CD45 expression,

21. The population of NK cells of claim 19, wherein CD45 (PTPRC) is knocked out, knocked down, or mutated by CRISPR-Cas9 or TALEN system.

22. The population of T cells of claim 18 or the population of NK cells of claim 21, wherein the CRISPR/CAS9 system comprises a gRNA targeted to a CD45 exon.

23. The population of T cells of claim 18 or the population of NK cells of claim 21, wherein the CRISPR/CAS9 system comprises a gRNA targeted to CD45 exon #3, CD45 exon #8, CD45 exon #12, or CD45 exon #25.

24. The population of T cells of claim 18 or the population of NK cells of claim 21, wherein the CRISPR/CAS9 system comprises a gRNA comprising SEQ ID NO: 37, 38, 39, 40, 41, 42, 43, 44, or 45, or a variant thereof having 1-5 nucleotide changes.

25. A method of treating a hematopoietic malignancy or hematopoietic disorder in a patient comprising administering a population of autologous or allogeneic human T or NK cells transduced by a vector comprising the nucleic acid molecule of claim 1, wherein the hematopoietic malignancy or hematopoietic disorder comprises cells expressing CD45, where in the CD45 (PTPRC) is knocked out, knocked down, or mutated in the human T or NK cells.

26. The method of claim 25, wherein the hematopoietic malignancy or hematopoietic disorder is any one or more of a leukemia, a lymphoma, a myeloma, a myeloid leukemia, a T cell leukemia, a T cell lymphoma, a B cell leukemia, a B cell lymphoma, AML, CML, ALL, multiple myeloma, sickle cell anemia, aplastic anemia, severe combined immunodeficiency, myelodysplastic syndromes, myeloproliferative neoplasms, histiocytic and dendritic cell neoplasms.

27. The method of claim 25, wherein the population of T cells or NK cells expressing the chimeric antigen receptor or the polypeptide is administered locally or systemically.

28. The method of claim 25, wherein the CD45-expressing cells are cancerous cells.

29. The method of claim 25, wherein the population of human T cells expressing the chimeric antigen receptor or the polypeptide is administered by single or repeat dosing.

30. A method of reducing or eliminating CD45-positive cells in a subject comprising administering a population of autologous or allogeneic human T or NK cells transduced by a vector comprising the nucleic acid molecule of claim 1, wherein the PTPRC is knocked out, knocked down, or mutated in the T or NK cells.

31. The method of claim 30, wherein the population of T cells or NK cells expressing the chimeric antigen receptor or the polypeptide is administered locally or systemically.

32. The method of claim 30, wherein the CD45-positive cells are cancerous cells or noncancerous cells.

33. The method of claim 30, wherein the population of human T cells expressing the chimeric antigen receptor or the polypeptide is administered by single or repeat dosing.

34. A method of hematological cell transplantation conditioning in a patient comprising administering a population of autologous or allogeneic human T or NK cells transduced by a vector comprising the nucleic acid molecule of claim 1, wherein the PTPRC is knocked out, knocked down, or mutated in the T or NK cells.

35. The method of claim 34, wherein the population of T cells or NK cells expressing the chimeric antigen receptor or the polypeptide is administered locally or systemically.

36. The method of claim 34, wherein CD45-expressing cells are reduced or eliminated.

37. The method of claim 34, wherein the population of human T cells expressing the chimeric antigen receptor or the polypeptide is administered by single or repeat dosing.

38. The method of claim 34, wherein the hematological cell transplantation conditioning precedes a hematopoietic cell transplantation.

39. The method of claim 38, wherein the hematopoietic cell transplantation is a bone marrow transplant.

40. A method of preparing CD45 CAR T cells comprising:
providing a population of autologous or allogeneic human T cells or NK cells
modifying the T cells or NK cells to reduce expression of CD45, and
introducing into the T cells or NK cells the nucleic acid molecule of claim 1.

41. The method of claim 40, wherein the T cells are at least about 80%, 85%, 90%, 95%, 96%, 97%, 98%, 99%, or 100% CD14 negative and at least about 80%, 85%, 90%, 95%, 96%, 97%, 98%, 99%, or 100% CD25 negative.

42. The method of claim 40, wherein the T cells comprise CD4+ T cells or CD8+ T cells or both.

43. A method of enhancing T cell proliferation in T cells expressing a CAR comprising knocking out, knocking down, or mutating the PTPRC gene in the T cells or NK cells thereby creating CD45− CAR T cells.

44. The method of claim 43, wherein there is less than 25%, 20%, 15%, 10%, 9% 8%, 7%, 6%, 5%, 4%, 3%, 2%, or 1% decrease in antigen-specific toxicity of the CD45-CAR T or NK cells cells compared the CD45+ CAR T cells or NK cells expressing the same CAR.

45. The population of T cells of claim 18 or the population of NK cells of claim 21, wherein the fratricide effect is less than about 10%, 9%, 8%, 7%, 6%, 5%, 4%, 3%, 2%, or 1%.


SEQUENCE LISTING (84 sequences)

SEQ ID NO: 1 - protein, 260 aa, Artificial Sequence
  source: /note=“Description of Artificial Sequence Synthetic polypeptide”
QVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSKISGGGGSGGGGSGGGGSGGGGSGGGGSSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIK

SEQ ID NO: 2 - protein, 10 aa, Artificial Sequence
  source: /note=“Description of Artificial Sequence Synthetic peptide”
GGGSSGGGSG

SEQ ID NO: 3 - protein, 12 aa, Artificial Sequence
  source: /note=“Description of Artificial Sequence Synthetic peptide”
ESKYGPPCPPCP

SEQ ID NO: 4 - protein, 12 aa, Homo sapiens
ESKYGPPCPSCP

SEQ ID NO: 5 - protein, 22 aa, Artificial Sequence
  source: /note=“Description of Artificial Sequence Synthetic peptide”
ESKYGPPCPPCPGGGSSGGGSG

SEQ ID NO: 6 - protein, 39 aa, Homo sapiens
IEVMYPPPYLDNEKSNGTIIHVKGKHLCPSPLFPGPSKP

SEQ ID NO: 7 - protein, 48 aa, Homo sapiens
AKPTTTPAPRPPTPAPTIASQPLSLRPEACRPAAGGAVHTRGLDFACD

SEQ ID NO: 8 - protein, 42 aa, Homo sapiens
PAPRPPTPAPTIASQPLSLRPEACRPAAGGAVHTRGLDFACD

SEQ ID NO: 9 - protein, 129 aa, Artificial Sequence
  source: /note=“Description of Artificial Sequence Synthetic polypeptide”
ESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 229 aa, Artificial Sequence
  source: /note=“Description of Artificial Sequence Synthetic polypeptide”
ESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 229 aa, Artificial Sequence
  source: /note=“Description of Artificial Sequence Synthetic polypeptide”
ESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 107 aa, Artificial Sequence
  source: /note=“Description of Artificial Sequence Synthetic polypeptide”
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 21 aa, Homo sapiens
LCYLLDGILFIYGVILTALFL

SEQ ID NO: 14 - protein, 27 aa, Homo sapiens
FWVLVVVGGVLACYSLLVTVAFIIFWV

SEQ ID NO: 15 - protein, 28 aa, Homo sapiens
MFWVLVVVGGVLACYSLLVTVAFIIFWV

SEQ ID NO: 16 - protein, 22 aa, Homo sapiens
MALIVLGGVAGLLLFIGLGIFF

SEQ ID NO: 17 - protein, 21 aa, Homo sapiens
IYIWAPLAGTCGVLLLSLVIT

SEQ ID NO: 18 - protein, 23 aa, Homo sapiens
IYIWAPLAGTCGVLLLSLVITLY

SEQ ID NO: 19 - protein, 24 aa, Homo sapiens
IYIWAPLAGTCGVLLLSLVITLYC

SEQ ID NO: 20 - protein, 27 aa, Homo sapiens
IISFFLALTSTALLFLLFFLTLRFSVV

SEQ ID NO: 21 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 41 aa, Homo sapiens
RSKRSRLLHSDYMNMTPRRPGPTRKHYQPYAPPRDFAAYRS

SEQ ID NO: 23 - protein, 41 aa, Homo sapiens
RSKRSRGGHSDYMNMTPRRPGPTRKHYQPYAPPRDFAAYRS

SEQ ID NO: 24 - protein, 42 aa, Homo sapiens
KRGRKKLLYIFKQPFMRPVQTTQEEDGCSCRFPEEEEGGCEL

SEQ ID NO: 25 - protein, 42 aa, Homo sapiens
ALYLLRRDQRLPPDAHKPPGGGSFRTPIQEEQADAHSTLAKI

SEQ ID NO: 26 - protein, 323 aa, Artificial Sequence
  source: /note=“Description of Artificial Sequence Synthetic polypeptide”
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 27 - protein, 24 aa, Artificial Sequence
  source: /note=“Description of Artificial Sequence Synthetic peptide”
LEGGGEGRGSLLTCGDVEENPGPR

SEQ ID NO: 28 - protein, 354 aa, Artificial Sequence
  source: /note=“Description of Artificial Sequence Synthetic polypeptide”
LVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 29 - protein, 695 aa, Artificial Sequence
  source: /note=“Description of Artificial Sequence Synthetic polypeptide”
MLLLVTSLLLCELPHPAFLLIPQVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSKISGGGGSGGGGSGGGGSGGGGSGGGGSSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - protein, 673 aa, Artificial Sequence
  source: /note=“Description of Artificial Sequence Synthetic polypeptide”
QVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSKISGGGGSGGGGSGGGGSGGGGSGGGGSSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - protein, 1077 aa, Artificial Sequence
  source: /note=“Description of Artificial Sequence Synthetic polypeptide”
MLLLVTSLLLCELPHPAFLLIPQVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSKISGGGGSGGGGSGGGGSGGGGSGGGGSSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMT

SEQ ID NO: 32 - protein, 123 aa, Artificial Sequence
  source: /note=“Description of Artificial Sequence Synthetic polypeptide”
QVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSKIS

SEQ ID NO: 33 - protein, 111 aa, Artificial Sequence
  source: /note=“Description of Artificial Sequence Synthetic polypeptide”
DIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIK

SEQ ID NO: 34 - protein, 4 aa, Artificial Sequence
  source: /note=“Description of Artificial Sequence Synthetic peptide”
GGGS

SEQ ID NO: 35 - protein, 5 aa, Artificial Sequence
  source: /note=“Description of Artificial Sequence Synthetic peptide”
GGGGS

SEQ ID NO: 36 - protein, 22 aa, Homo sapiens
MLLLVTSLLLCELPHPAFLLIP

SEQ ID NO: 37 - RNA, 20 nt, Artificial Sequence
  source: /note=“Description of Artificial Sequence Synthetic oligonucleotide”
auauuaauucuuaccagugg

SEQ ID NO: 38 - RNA, 20 nt, Artificial Sequence
  source: /note=“Description of Artificial Sequence Synthetic oligonucleotide”
acuccaucuaagccaacaug

SEQ ID NO: 39 - RNA, 20 nt, Artificial Sequence
  source: /note=“Description of Artificial Sequence Synthetic oligonucleotide”
cuucuacaaaaaauaaucug

SEQ ID NO: 40 - RNA, 17 nt, Artificial Sequence
  source: /note=“Description of Artificial Sequence Synthetic oligonucleotide”
gugcugguguugggcgc

SEQ ID NO: 41 - RNA, 20 nt, Artificial Sequence
  source: /note=“Description of Artificial Sequence Synthetic oligonucleotide”
uuaugaaaugaucuuugagg

SEQ ID NO: 42 - RNA, 20 nt, Artificial Sequence
  source: /note=“Description of Artificial Sequence Synthetic oligonucleotide”
aaaauaaucugaggcucucc

SEQ ID NO: 43 - RNA, 20 nt, Artificial Sequence
  source: /note=“Description of Artificial Sequence Synthetic oligonucleotide”
auaguaugcaugucaagugu

SEQ ID NO: 44 - RNA, 20 nt, Artificial Sequence
  source: /note=“Description of Artificial Sequence Synthetic oligonucleotide”
gggccauuacggucccuggg

SEQ ID NO: 45 - RNA, 19 nt, Artificial Sequence
  source: /note=“Description of Artificial Sequence Synthetic oligonucleotide”
gugcugguguugggcgcac

SEQ ID NO: 46 - protein, 121 aa, Artificial Sequence
  source: /note=“Description of Artificial Sequence Synthetic polypeptide”
QVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSK

SEQ ID NO: 47 - protein, 5 aa, Artificial Sequence
  source: /note=“Description of Artificial Sequence Synthetic peptide”
RYWMS

SEQ ID NO: 48 - protein, 17 aa, Artificial Sequence
  source: /note=“Description of Artificial Sequence Synthetic peptide”
EINPTSSTINFTPSLKD

SEQ ID NO: 49 - protein, 12 aa, Artificial Sequence
  source: /note=“Description of Artificial Sequence Synthetic peptide”
GNYYRYGDAMDY

SEQ ID NO: 50 - protein, 15 aa, Artificial Sequence
  source: /note=“Description of Artificial Sequence Synthetic peptide”
RASKSVSTSGYSYLH

SEQ ID NO: 51 - protein, 7 aa, Artificial Sequence
  source: /note=“Description of Artificial Sequence Synthetic peptide”
LASNLES

SEQ ID NO: 52 - protein, 14 aa, Artificial Sequence
  source: /note=“Description of Artificial Sequence Synthetic peptide”
QHSRELPFTFGSGT

SEQ ID NO: 53 - protein, 21 aa, Homo sapiens
PFFFCCFIAVAMGIRFIIMVT

SEQ ID NO: 54 - protein, 120 aa, Homo sapiens
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 55 - DNA, 284 bp, Artificial Sequence
  source: /note=“Description of Artificial Sequence Synthetic polynucleotide”
cagctcgctttcttgctgtccaatttctattaaaggttcctttgttccctaagtccaactactaaactgggggatattatgaagggccttgagcatctggattctgcctaataaaaaacatttattttcattgctgcgtcgagagctcgctttcttgctgtccaatttctattaaaggttcctttgttccctaagtccaactactaaactgggggatattatgaagggccttgagcatctggattctgcctaataaaaaacatttattttcattgctgcctcga

SEQ ID NO: 56 - RNA, 150 nt, Artificial Sequence
  source: /note=“Description of Artificial Sequence Synthetic polynucleotide”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 57 - DNA, 19 bp, Artificial Sequence
  source: /note=“Description of Artificial Sequence Synthetic oligonucleotide”
taatacgactcactatagg

SEQ ID NO: 58 - DNA, 2560 bp, Artificial Sequence
  source: /note=“Description of Artificial Sequence Synthetic polynucleotide”
taatacgactcactatagggaaagctcgagtatgttgctgctggttacatctctgctgctgtgcgagctgccccatcctgcctttctgctgattcctcaggtgcagctggtggaatctggcggaggacttgttcagcctggcggctctctgaagctgtcttgtgccgccagcggcttcgacttcagcagatactggatgagctgggtccgacaggcccctggcaaaggactggaatggatcggcgagatcaaccccacctccagcaccatcaacttcacccctagcctgaaggacaaggtgttcatcagccgggacaacgccaagaacaccctgtacctgcagatgagcaaagtgcggagcgaggacacagccctgtactactgtgccagaggcaactactacagatacggcgacgccatggactactggggccagggaacaagcgtgaccgtgtctaagatttctggcggcggaggaagcggaggcggaggatccggcggtggtggtagtggtggtggcggttcaggtggcggtggaagctctgatatcgtgctgacacagagccccgcttctctggccgtttctctgggacagagagccaccatcagctgcagagccagcaagagcgtgtccacaagcggctacagctacctgcactggtatcagcaaaagcccggccagcctcctaagctgctgatctacctggccagcaacctggaaagcggagtgcctgccagattttctggcagcggctctggcaccgacttcaccctgaatatccatcctgtggaagaagaggacgccgccacttactactgccagcacagcagagaactgcccttcacattcggcagcggcaccaagctggaaatcaaagagtctaagtacggccctccttgtcctccatgtcctgctccagagtttgaaggcggcccttccgtgttcctgtttcctccaaagcctaaggacaccctgatgatcagcaggacccctgaggtgacctgcgtggtggtggacgtgagccaggaagatcccgaggtccagttcaattggtacgtggacggcgtggaagtgcacaacgccaagaccaagcccagagaggaacagttccagagcacctaccgggtggtgtctgtgctgaccgtgctgcaccaggactggctgaacggcaaagaatacaagtgcaaggtgtccaacaagggcctgcccagcagcatcgaaaagaccatcagcaaggccaagggccagcctcgcgagccccaggtgtacaccctgcctccctcccaggaagagatgaccaagaaccaggtgtccctgacctgcctggtgaagggcttctaccccagcgacatcgccgtggagtgggagagcaacggccagcctgagaacaactacaagaccacccctcccgtgctggacagcgacggcagcttcttcctgtacagccggctgaccgtggacaagagccggtggcaggaaggcaacgtctttagctgcagcgtgatgcacgaggccctgcacaaccactacacccagaagagcctgagcctgtccctgggcaagatgttctgggtgctggtggtggtgggcggggtgctggcctgctacagcctgctggtgacagtggccttcatcatcttttgggtgcggagcaagcggagcagaggcggccacagcgactacatgaacatgacccccagacggcctggccccacccggaagcactaccagccctacgccccacccagggactttgccgcctaccggtccggcggagggcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggtgacagctcgctttcttgctgtccaatttctattaaaggttcctttgttccctaagtccaactactaaactgggggatattatgaagggccttgagcatctggattctgcctaataaaaaacatttattttcattgctgcgtcgagagctcgctttcttgctgtccaatttctattaaaggttcctttgttccctaagtccaactactaaactgggggatattatgaagggccttgagcatctggattctgcctaataaaaaacatttattttcattgctgcctcgacgaattcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 59 - protein, 400 aa, Artificial Sequence
  source: /note=“Description of Artificial Sequence Synthetic polypeptide”
MLEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 60 - protein, 677 aa, Artificial Sequence
  source: /note=“Description of Artificial Sequence Synthetic polypeptide”
MLEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTEGRGSLLTCGDVEENPGPQVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSKISGGGGSGGGGSGGGGSGGGGSGGGGSSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIK

SEQ ID NO: 61 - protein, 677 aa, Artificial Sequence
  source: /note=“Description of Artificial Sequence Synthetic polypeptide”
QVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSKISGGGGSGGGGSGGGGSGGGGSGGGGSSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKEGRGSLLTCGDVEENPGPMLEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKT

SEQ ID NO: 62 - protein, 595 aa, Artificial Sequence
  source: /note=“Description of Artificial Sequence Synthetic polypeptide”
MLLLVTSLLLCELPHPAFLLIPQVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSKISGGGGSGGGGSGGGGSGGGGSGGGGSSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 63 - protein, 573 aa, Artificial Sequence
  source: /note=“Description of Artificial Sequence Synthetic polypeptide”
QVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSKISGGGGSGGGGSGGGGSGGGGSGGGGSSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 64 - protein, 977 aa, Artificial Sequence
  source: /note=“Description of Artificial Sequence Synthetic polypeptide”
MLLLVTSLLLCELPHPAFLLIPQVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSKISGGGGSGGGGSGGGGSGGGGSGGGGSSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMT

SEQ ID NO: 65 - protein, 767 aa, Artificial Sequence
  source: /note=“Description of Artificial Sequence Synthetic polypeptide”
MLLLVTSLLLCELPHPAFLLIPQVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSKISGGGGSGGGGSGGGGSGGGGSGGGGSSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKPFFFCCFIAVAMGIRFIIMVTWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 66 - protein, 745 aa, Artificial Sequence
  source: /note=“Description of Artificial Sequence Synthetic polypeptide”
QVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSKISGGGGSGGGGSGGGGSGGGGSGGGGSSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKPFFFCCFIAVAMGIRFIIMVTWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 67 - protein, 1149 aa, Artificial Sequence
  source: /note=“Description of Artificial Sequence Synthetic polypeptide”
MLLLVTSLLLCELPHPAFLLIPQVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSKISGGGGSGGGGSGGGGSGGGGSGGGGSSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKPFFFCCFIAVAMGIRFIIMVTWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMT

SEQ ID NO: 68 - protein, 667 aa, Artificial Sequence
  source: /note=“Description of Artificial Sequence Synthetic polypeptide”
MLLLVTSLLLCELPHPAFLLIPQVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSKISGGGGSGGGGSGGGGSGGGGSGGGGSSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKPFFFCCFIAVAMGIRFIIMVTWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 69 - protein, 645 aa, Artificial Sequence
  source: /note=“Description of Artificial Sequence Synthetic polypeptide”
QVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSKISGGGGSGGGGSGGGGSGGGGSGGGGSSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKPFFFCCFIAVAMGIRFIIMVTWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 70 - protein, 1049 aa, Artificial Sequence
  source: /note=“Description of Artificial Sequence Synthetic polypeptide”
MLLLVTSLLLCELPHPAFLLIPQVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSKISGGGGSGGGGSGGGGSGGGGSGGGGSSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKPFFFCCFIAVAMGIRFIIMVTWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMT

SEQ ID NO: 71 - protein, 580 aa, Artificial Sequence
  source: /note=“Description of Artificial Sequence Synthetic polypeptide”
MLLLVTSLLLCELPHPAFLLIPQVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSKISGGGGSGGGGSGGGGSGGGGSGGGGSSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKPAPRPPTPAPTIASQPLSLRPEACRPAAGGAVHTRGLDFACDPFFFCCFIAVAMGIRFIIMVTWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 72 - protein, 558 aa, Artificial Sequence
  source: /note=“Description of Artificial Sequence Synthetic polypeptide”
QVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSKISGGGGSGGGGSGGGGSGGGGSGGGGSSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKPAPRPPTPAPTIASQPLSLRPEACRPAAGGAVHTRGLDFACDPFFFCCFIAVAMGIRFIIMVTWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 73 - protein, 962 aa, Artificial Sequence
  source: /note=“Description of Artificial Sequence Synthetic polypeptide”
MLLLVTSLLLCELPHPAFLLIPQVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSKISGGGGSGGGGSGGGGSGGGGSGGGGSSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKPAPRPPTPAPTIASQPLSLRPEACRPAAGGAVHTRGLDFACDPFFFCCFIAVAMGIRFIIMVTWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMT

SEQ ID NO: 74 - protein, 502 aa, Artificial Sequence
  source: /note=“Description of Artificial Sequence Synthetic polypeptide”
MLLLVTSLLLCELPHPAFLLIPQVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSKISGGGGSGGGGSGGGGSGGGGSGGGGSSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 75 - protein, 480 aa, Artificial Sequence
  source: /note=“Description of Artificial Sequence Synthetic polypeptide”
QVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSKISGGGGSGGGGSGGGGSGGGGSGGGGSSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 76 - protein, 884 aa, Artificial Sequence
  source: /note=“Description of Artificial Sequence Synthetic polypeptide”
MLLLVTSLLLCELPHPAFLLIPQVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSKISGGGGSGGGGSGGGGSGGGGSGGGGSSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMT

SEQ ID NO: 77 - protein, 590 aa, Artificial Sequence
  source: /note=“Description of Artificial Sequence Synthetic polypeptide”
MLLLVTSLLLCELPHPAFLLIPQVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSKISGGGGSGGGGSGGGGSGGGGSGGGGSSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 78 - protein, 568 aa, Artificial Sequence
  source: /note=“Description of Artificial Sequence Synthetic polypeptide”
QVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSKISGGGGSGGGGSGGGGSGGGGSGGGGSSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 79 - protein, 972 aa, Artificial Sequence
  source: /note=“Description of Artificial Sequence Synthetic polypeptide”
MLLLVTSLLLCELPHPAFLLIPQVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSKISGGGGSGGGGSGGGGSGGGGSGGGGSSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMT

SEQ ID NO: 80 - protein, 690 aa, Artificial Sequence
  source: /note=“Description of Artificial Sequence Synthetic polypeptide”
MLLLVTSLLLCELPHPAFLLIPQVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSKISGGGGSGGGGSGGGGSGGGGSGGGGSSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 81 - protein, 668 aa, Artificial Sequence
  source: /note=“Description of Artificial Sequence Synthetic polypeptide”
QVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSKISGGGGSGGGGSGGGGSGGGGSGGGGSSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 82 - protein, 1072 aa, Artificial Sequence
  source: /note=“Description of Artificial Sequence Synthetic polypeptide”
MLLLVTSLLLCELPHPAFLLIPQVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSKISGGGGSGGGGSGGGGSGGGGSGGGGSSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMT

SEQ ID NO: 83 - protein, 40 aa, Artificial Sequence
  source: /note=“Description of Artificial Sequence Synthetic polypeptide”
  SITE (1)..(40): /note=“This sequence may encompass 1-10 ′Gly Gly Gly Ser′ repeating units”
GGGSGGGSGGGSGGGSGGGSGGGSGGGSGGGSGGGSGGGS

SEQ ID NO: 84 - protein, 50 aa, Artificial Sequence
  source: /note=“Description of Artificial Sequence Synthetic polypeptide”
  SITE (1)..(50): /note=“This sequence may encompass 1-10 ′Gly Gly Gly Gly Ser′ repeating units”
GGGGSGGGGSGGGGSGGGGSGGGGSGGGGSGGGGSGGGGSGGGGSGGGGS